(19) United States
(12) Patent Application Publication
(10) Pub. No.: US 2026/0311882 A1
(43) Pub. Date: Oct. 8, 2026

(54) INFORMATION PROCESSING SYSTEM AND INFORMATION PROCESSING METHOD

(71) Applicant: Paramount Bed Co., Ltd., Tokyo (JP)

(72) Inventors: Akira NAKAI, Koto-ku (JP); Junichi TAMURA, Koto-ku (JP)

(73) Assignee: Paramount Bed Co., Ltd., Tokyo (JP)

(21) Appl. No.: 19/349,484

(22) Filed: Oct. 3, 2025

(30) Foreign Application Priority Data
Apr. 2, 2025 (JP) 2025-060905

(51) Int. Cl. G16H 50/30

(52) U.S. Cl. CPC G16H 50/30

(57) ABSTRACT

An information processing system includes: a posture information acquisition unit configured to acquire posture information; a health information acquisition unit configured to acquire health information; a memory configured to store the posture information and the health information in association with each other; and a processing unit configured to determine a state of a user based on the health information, and perform at least one of (1) a process of obtaining risky posture information based on the posture information associated with the health information in which the state of the user is determined as a first state with a worsening risk being present, and (2) a process of obtaining safety posture information based on the posture information associated with the health information in which the state of the user is determined as a second state with the worsening risk being relatively low.

### CROSS-REFERENCE TO RELATED APPLICATIONS

This application is based upon and claims the benefit of priority from Japanese Patent Application No.2025-060905, filed on Apr. 2, 2025; the entire contents of which are incorporated herein by reference.

### FIELD

The present disclosure relates to an information processing system, an information processing method, and the like.

### BACKGROUND

In the related art, a method for presenting information of a patient in a system including a terminal provided in the vicinity of a bed is known (for example, JP2017-18379A).

### BRIEF DESCRIPTION OF THE DRAWINGS

FIG. 1 is a diagram illustrating a configuration example of an information processing system.

FIG. 2 is a diagram illustrating a configuration example of a bed.

FIG. 3 is a diagram illustrating a configuration example of a bedside terminal device.

FIG. 4 is a diagram illustrating a configuration example of a server system.

FIG. 5 is a diagram illustrating a configuration example of a terminal device.

FIG. 6 is a flowchart illustrating a process of obtaining risky posture information.

FIG. 7A is a diagram illustrating an example of a positional relationship between a bed and an imaging device.

FIG. 7B is a diagram illustrating an example of a positional relationship between the bed and the imaging device.

FIG. 8 is a diagram illustrating an example of data stored in a memory.

FIG. 9A is a diagram illustrating an example of statistic values obtained for each posture.

FIG. 9B is a diagram illustrating an example of statistic values of SpO2 for each posture.

FIG. 9C is a diagram illustrating an example of statistic values of heartbeat rate for each posture.

FIG. 9D is a diagram illustrating an example of statistic values of respiratory rate for each posture.

FIG. 10 is a flowchart illustrating a process of obtaining safety posture information.

FIG. 11 is a diagram illustrating an example of data stored in the memory.

FIG. 12 is a graph showing time-series changes in vital information (respiratory rate).

FIG. 13 is a diagram illustrating an example of data acquired from a target user who is a pregnant female.

FIG. 14A is a diagram illustrating an example of statistic values of blood pressure values in a supine position according to a gestation cycle.

FIG. 14B is a diagram illustrating a relationship between gestation cycle and systolic blood pressure.

FIG. 14C is a diagram illustrating a relationship between gestation cycle and diastolic blood pressure.

FIG. 15 is a flowchart illustrating processing using sleep information.

FIG. 16 is a flowchart illustrating processing using feedback information.

FIG. 17 is a flowchart illustrating processing using defecation information.

FIG. 18 is a graph showing correspondence between a time of each posture during a sleep on the previous day and presence or absence of defecation.

FIG. 19 is a diagram illustrating an example of a display screen including list data in which posture information and time information are associated with each other for vital information in which the state is determined to be bad.

FIG. 20A is a diagram illustrating an example of a screen displaying an analysis result.

FIG. 20B is a diagram illustrating an example of a screen displaying an analysis result.

FIG. 20C is a diagram illustrating an example of a screen displaying an analysis result.

FIG. 21 is a flowchart illustrating notification processing.

FIG. 22 is a flowchart illustrating automatic control of a bed or the like.

### DETAILED DESCRIPTION

One or more embodiments are now described with reference to the drawings, wherein like reference numerals are used to refer to like elements throughout. In the following description, for purposes of explanation, numerous specific details are set forth in order to provide a thorough understanding of the various embodiments. It is evident, however, that the various embodiments can be practiced without these specific details (and without applying to any particular networked environment or standard).

As used in this disclosure, in some embodiments, the terms "component," "system" and the like are intended to refer to, or comprise, a computer-related entity or an entity related to an operational apparatus with one or more specific functionalities, wherein the entity can be either hardware, or a combination of hardware and software in execution.

One or more components may reside within a process and/or thread of execution and a component may be localized on one computer and/or distributed between two or more computers. In addition, these components can execute from various computer readable media having various data structures stored thereon. The components may communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software application or firmware application executed by a processor, wherein the processor can be internal or external to the apparatus and executes at least a part of the software or firmware application. As yet another example, a component can be an apparatus that provides specific functionality through electronic components without mechanical parts, the electronic components can comprise a processor therein to execute software stored on a non-transitory electronic memory or firmware that confers at least in part the functionality of the electronic components. While various components have been illustrated as separate components, it will be appreciated that multiple components can be implemented as a single component, or a single component can be implemented as multiple components, without departing from example embodiments. Further, the various embodiments can be implemented as a method, apparatus or article of manufacture using standard programming and/or engineering techniques to produce software, firmware, hardware or any combination thereof to control a computer to implement the disclosed subject matter. The term "article of manufacture" as used herein is intended to encompass a computer-readable (or machine-readable) device or computer-readable (or machine-readable) storage/communications media having a computer program stored thereon. For example, computer readable storage media can comprise, but are not limited to, magnetic storage devices (e.g., hard disk, floppy disk, magnetic strips), optical disks (e.g., compact disk (CD), digital versatile disk (DVD)), smart cards, and flash memory devices (e.g., card, stick, key drive). Of course, those skilled in the art will recognize many modifications can be made to this configuration without departing from the scope or spirit of the various embodiments.

In addition, the words "example" and "exemplary" are used herein to mean serving as an instance or illustration. Any embodiment or design described herein as "example" or "exemplary" is not necessarily to be construed as preferred or advantageous over other embodiments or designs. Rather, use of the word example or exemplary is intended to present concepts in a concrete fashion. As used in this application, the term "or" is intended to mean an inclusive "or" rather than an exclusive "or". That is, unless specified otherwise or clear from context, "X employs A or B" is intended to mean any of the natural inclusive permutations. That is, if X employs A; X employs B; or X employs both A and B, then "X employs A or B" is satisfied under any of the foregoing instances. In addition, the articles "a" and "an" as used in this application and the appended claims should generally be construed to mean "one or more" unless specified otherwise or clear from context to be directed to a singular form.

Embodiments described herein can be exploited in substantially any wireless communication technology, comprising, but not limited to, wireless fidelity (Wi-Fi), global system for mobile communications (GSM), universal mobile telecommunications system (UMTS), worldwide interoperability for microwave access (WiMAX), enhanced general packet radio service (enhanced GPRS), third generation partnership project (3GPP) long term evolution (LTE), third generation partnership project 2 (3GPP2) ultra mobile broadband (UMB), high speed packet access (HSPA), Z-Wave, Zigbee and other 802.XX wireless technologies and/or legacy telecommunication technologies.

In general, one aspect of the present application is an information processing system including:
a posture information acquisition unit configured to acquire posture information indicating a posture of a user;
a health information acquisition unit configured to acquire health information indicating a health condition of the user;
a memory configured to store the posture information and the health information in association with each other; and
a processing unit configured to determine a state of the user based on the health information, and perform at least one of
   (1) a process of obtaining risky posture information based on the posture information associated with the health information in which the state of the user is determined as a first state with a worsening risk being present, and
   (2) a process of obtaining safety posture information based on the posture information associated with the health information in which the state of the user is determined as a second state with the worsening risk being relatively low.

Another aspect of the present application is an information processing method performed by an information processing system, the method comprising:
the information processing system acquiring posture information indicating a posture of a user,
the information processing system acquiring health information of the user,
the information processing system storing the posture information and the health information in association with each other, the information processing system determining a state of the user based on the health information, and
the information processing system performing at least one of
   (1) a process of obtaining risky posture information based on the posture information associated with the health information in which the state of the user is determined as a first state with a worsening risk being present, and
   (2) a process of obtaining safety posture information based on the posture information associated with the health information in which the state of the user is determined as a second state with the worsening risk being relatively low.

Provided is an information processing system, a control method, and the like suitable for a field such as care for a user.

Hereinafter, the present embodiment will be described with reference to the drawings. Throughout the drawings, the same or similar components are assigned with the same reference signs, and a redundant description thereof will be omitted. Note that, the embodiment to be described below is not intended to unjustly limit the contents described in the scope of claims. In addition, not all the configurations to be described in the embodiment are necessarily essential elements of the disclosure.

### 1. System Configuration Example

It is known that the orientation and posture of the body of a user significantly affect the health condition. Specifically, during a sleep in which the same posture is kept for a long time, the user may unconsciously take a risky posture for a long time. The user here may be a patient suffering from any disease or a care receiver who receives care by a care giver in a care facility, at home, or the like. Further, the user may include a person who has no problem in the health condition. People around the user are also less likely to notice that the user takes a risky posture for a long time during a sleep or the like. The people around may be a health professional such as a medical doctor, a care worker, and a nurse (hereinafter referred to as a staff member) or may be a family member of the user.

As the risk here, for example, in the case of a patient having a difference in function between the left and right lungs, there is a known risk that the blood oxygen saturation (SpO2) is likely to be lowered when the patient takes a lateral position with the side of the lung having the lower function facing down. In the case of a pregnant female in a late gestation (e.g., 28 to 40 weeks), if she sleeps in a supine position, there is a possibility that the risk of supine hypotension syndrome in which the blood pressure is lowered, and tachycardia, vomiting, cold sweat, dizziness, and the like are caused is increased. For a patient needing a surgical treatment, it is conceivable that the patient may feel discomfort due to pain, numbness, or the like depending on the orientation or posture of the body according to effects such as an injured site, a scar from surgery, pressure ulcer, contracture of a joint, or the like, and when such a risky posture continues for a long time and a stress is continuously applied, an effect such as shallow breathing may be caused. In addition, the risks due to postures are not limited thereto.

An information processing system according to the embodiment monitors posture information indicating the posture of a patient and health information indicating the health condition of the patient, and determines a risky posture that may lead to worsening of the condition. Alternatively, the information processing system according to the embodiment may perform a process of determining a safety posture that leads to improvement of the condition (or prevention of worsening) by monitoring the posture information and the health information. By reflecting determined risky posture and safety posture into subsequent nursing-care/care plan, it is possible to reduce risks of worsening of the health condition due to postures. Hereinafter, the method of the embodiment will be described in detail. Note that the health information may be, for example, vital information indicating the state of biological activity, and the vital information may be, for example, sensing results such as respiratory rate, heartbeat rate, SpO2, and the like. In addition, the health information may include sleep information, excretion information, feedback information, and the like, which will be described later.

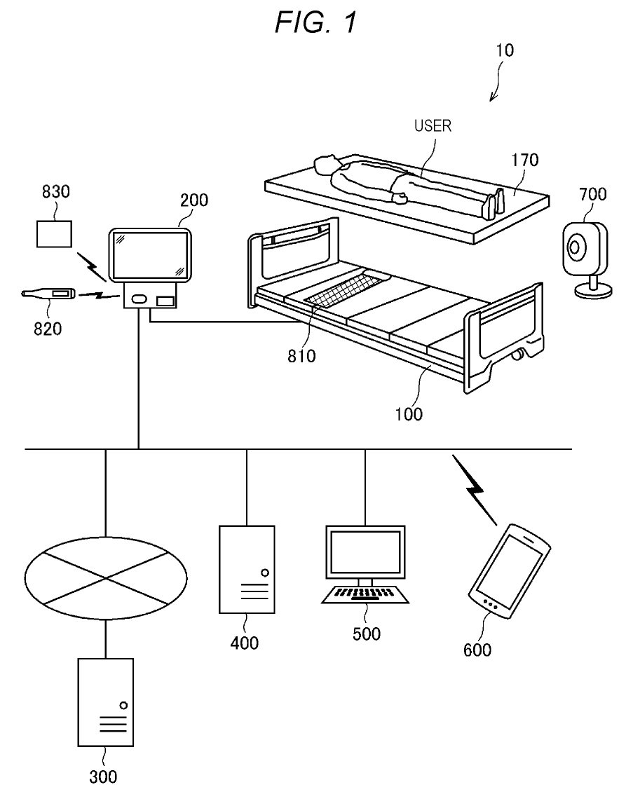

FIG. 1 is a diagram illustrating a configuration example of an information processing system 10 according to the embodiment. The information processing system 10 includes a bed 100, a bedside terminal device 200, a server system 300, an electronic medical record server 400, a station terminal device 500, and a terminal device 600 (mobile terminal device). In addition, the information processing system 10 may include an imaging device 700, a seat sensor 810, a measuring device 820, an authentication card 830, and the like. However, the configuration of the information processing system 10 is not limited to the example in FIG. 1, but various modifications such as omitting a part of the configuration, adding another configuration, or the like can be made.

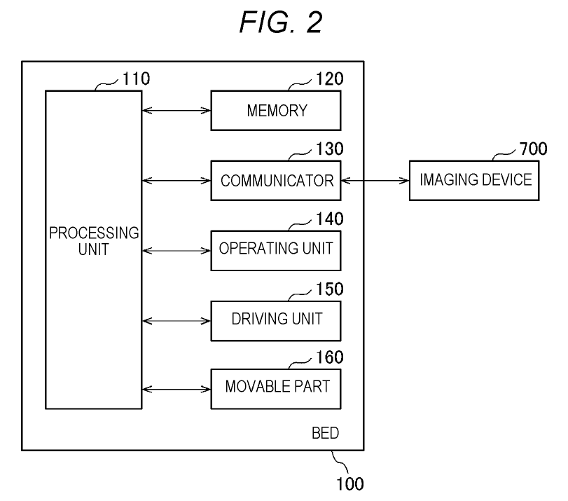

For example, the information processing system 10 according to the embodiment corresponds to the server system 300 in FIG. 1, and the other components may be external devices that are directly or indirectly connected to the information processing system 10 of the embodiment. Further, the information processing system 10 according to the embodiment may be implemented through distributed processing by two or more devices among the devices illustrated in FIG. 1. In addition, among the components illustrated in FIG. 1, the bedside terminal device 200 may be omitted. For example, a part or all of the processing executed by the bedside terminal device 200 that will be described below may be executed by another device such as the terminal device 600. Moreover, modifications such as the omission and addition of the configuration can be made similarly in FIG. 2 or the like that will be described below.

The bed 100 is bedding used by the user. For example, a mattress 170 is placed on the bed 100, and the user lies down on the mattress 170. The imaging device 700 and the seat sensor 810 are provided in the vicinity of the bed 100. The imaging device 700 is, for example, a camera fixed to a frame of the bed 100 or the like, but may be fixed to a wall surface of a hospital room or the like. The seat sensor 810 is, for example, a sheet-shaped or plate-shaped device that is provided between the bed 100 and the mattress 170.

The imaging device 700 outputs an imaged image using a sensor such as a charged-coupled device (CCD) image sensor or a complementary metal-oxide-semiconductor image sensor. The imaged image here may be a moving image or a still image. The imaging device 700 is provided in the vicinity of the bed 100 to image a patient present in a hospital room or a living room.

The imaging device 700 may include a posture determination unit that determines the position and posture of the user on the bed 100 based on an imaged image. The posture determination unit sequentially performs posture determination based on imaged images input in a time-series manner and outputs the determination results as the posture information. Note that the imaging device is a device that outputs an imaged image, and the posture determination based on the imaged image may be executed in another device such as the server system 300.

The seat sensor 810 is a device that senses information about patient's sleeping. The seat sensor 810 includes a pressure sensor (e.g., a pneumatic sensor) that outputs a pressure value. When the user goes to bed, the seat sensor 810 detects body vibration (body movement, vibration) of the user via the mattress 170. The seat sensor 810 obtains, based on the body vibration detected, information on a respiratory rate, a heartbeat rate, an activity amount, a posture, awake/sleeping, and bed departure/bed presence. Note that, the seat sensor 810 may output sensor data indicating the body vibration, and another device such as the server system 300 may execute a process of obtaining information such as a respiratory rate based on the sensor data. Hereinafter, an example in which the seat sensor 810 outputs information such as a respiratory rate will be described.

For example, the seat sensor 810 may analyze the periodicity of the body movement, and calculate a respiratory rate and a heartbeat rate from peak frequencies. The analysis of the periodicity is a Fourier transform, for example. The respiratory rate is the number of times of respiration per unit time. The heartbeat rate is the number of heartbeats per unit time. The unit time is one minute, for example. In addition, the seat sensor 810 may detect body vibration per sampling unit time and output the number of times of the body vibration detected as an activity amount. Moreover, since a pressure value to be detected decreases in the bed departure of the user as compared with in the bed presence, the seat sensor 810 may determine bed departure or bed presence based on the pressure value and a time-series changes in the pressure values. However, the method of determining bed departure or bed presence is not limited to the above, and various modifications such as detection of vibration can be made. Moreover, the seat sensor 810 may determine non REM sleep or REM sleep, or determine a sleep depth. The determination relating to a sleep may be executed based on a respiratory rate and a heartbeat rate, may be executed based on the amount of body movement (e.g., an activity amount), or may be executed using both of them.

The seat sensor 810 outputs vital information (information such as respiration and heartbeat) indicating the state of the biological activity of a patient, and sleep information related to a sleep (information such as sleeping/awake, a sleep depth, and bed departure/bed presence) to the server system 300. Note that the seat sensor 810 may output a sensing result to the server system 300 via the bedside terminal device 200, or may output a sensing result to the server system 300 without via the bedside terminal device 200.

A device equipped with a load sensor may be used as the bed 100. In that case, the bed 100 may output at least one of the vital information and the sleep information of a patient. In addition, the bed 100 may output information indicating a change in the position of the center of gravity of a patient, or information indicating sleeping posture. Further, the seat sensor 810 and the bed 100 including a load sensor may be used in combination.

The bedside terminal device 200 is a device having a function of notifying the state of a patient (notification function), and may be connected to the seat sensor 810 or may be connected to another device included in the information processing system 10 via a network.

For example, the bedside terminal device 200 may include a display device and a connection device. The display device is, for example, a tablet-type display terminal and executes displaying of various information and reception of input of various operations. The connection device is a hub device for connecting the display device and various devices. For example, the connection device may sequentially acquire the vital information of a patient by being connected to the seat sensor 810. Further, the connection device may receive the vital information from various measuring devices 820 (e.g., a thermometer), or may receive the vital information from a device worn by a patient (e.g., a wristwatch-type wearable measuring device). In addition, the connection device may perform authentication processing (e.g., authentication processing for a patient and login processing for a nurse or the like) by reading the authentication card 830. Note that the connection device may execute the authentication processing using near field communication (NFC) which is an example of near field wireless communication, or may execute the authentication processing in another method using a barcode, infrared ray, an IC tag, or the like. For example, by logging in, a staff member (a nurse, a medical doctor, or a care staff member) can check values of the vital information and notification contents, and register the contents thereof into an electronic medical record on an as-needed basis.

The network is connected with the server system 300, the electronic medical record server 400, the station terminal device 500, and the terminal device 600, for example.

The server system 300 is a server that provides various services, and may be connected to a LAN in a medical facility, or may be provided outside via the Internet.

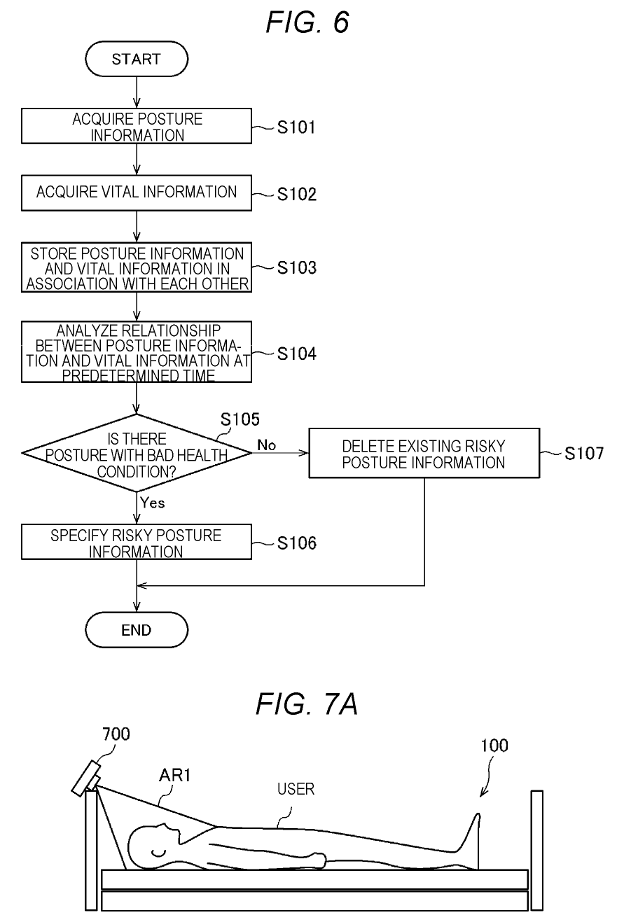

The server system 300 may be one server, or may include multiple servers. For example, the server system 300 may include a database server and an application server. The database server stores various types of data such as posture information, vital information, and sleep information. The application server performs processing that will be described below using FIGS. 6, 10, 15 to 17, and 21 to 22, and the like. Note that, the multiple servers here may be physical servers or may be virtual servers. In a case where a virtual server is used, the virtual server may be provided in one physical server, or may be disposed in a distributed manner to multiple physical servers. As described above, the specific configuration of the server system 300 of the embodiment can be modified in various ways.

The electronic medical record server 400 is a server that stores information of an electronic medical record related to a patient. The electronic medical record server 400 is generally a server that is connected to a network inside a medical facility, but may be an external cloud server, for example. The electronic medical record may include various types of information. The electronic medical record includes, for example, basic information, a medical history, diagnosis information, medication information, an inspection result, medical examination information, treatment information, a rehabilitation history, a preventive vaccination history, lifestyle information, a family history, written consent and signature, insurance and medical relations, medical team information, social background, mental health, an emergency response plan, medical resource history, communication with family, infection control measures, risk assessment, doctor's instruction information, a measurement result, a response history, a nurse call history, a rehabilitation care history, a changing hospital and referral history, a recuperation instruction history, a patient complaint and major complaint history, patient activity information, blood glucose level information, diet treatment information, drinking water information, cardiac electrogram information, pulse information, consultation information of multiple departments, cooperation information between departments, a record of activity moving image, moving image file storage information, response of doctor/nurse based on moving image, respiratory rate information, arterial blood oxygen saturation information, excretion information, room temperature and humidity information, and the like. However, the above are merely specific examples of information included in an electronic medical record, and various modifications such as omitting a part of information or adding other information can be made.

The electronic medical record of the embodiment may include another system. The other system includes, for example, a nursing care support system, a rehabilitation section system, a severe injury section system, and a nutrition section system.

The station terminal device 500 is a terminal device provided at a nurse station or a control room. A staff member can grasp the state of the bedside terminal device 200 from a location different from a hospital room or a living room by using the station terminal device 500. The state of the bedside terminal device 200 corresponds to the state of a patient corresponding to the bedside terminal device 200.

The terminal device 600 is a mobile device used by a staff member such as a medical doctor or a nurse. The terminal device 600 for a staff member is connected to a network (e.g., a LAN) by wireless communication. The staff member can confirm the information of the bedside terminal device 200 at each location in a medical facility by using the terminal device 600.

At least one of the station terminal device 500 and the terminal device 600 may execute a process of notifying (informing) a staff member of the state of a patient. The notification processing executed by the station terminal device 500 and/or the terminal device 600 may be executed with the bedside terminal device 200 as a starting point or may be executed with the server system 300 as a starting point.

Next, a configuration example of each device included in the information processing system 10 will be described using FIGS. 2 to 5.

FIG. 2 is a diagram illustrating a configuration example of the bed 100. The bed 100 includes, for example, a processing unit 110, a memory 120, a communicator 130, an operating unit 140, a driving unit 150, and a movable part 160. The bed 100 may include a load sensor or the like that is not illustrated in FIG. 2.

The processing unit 110 in the embodiment is implemented by hardware described below. The hardware can include at least one of a circuit that processes digital signals and a circuit that processes analog signals. For example, the hardware may include one or multiple circuit devices and one or multiple circuit elements mounted on a circuit board. The one or multiple circuit devices are, for example, an integrated circuit (IC) and a field-programmable gate array (FPGA). Examples of the one or multiple circuit elements include a resistor and a capacitor.

Moreover, the processing unit 110 may be implemented by processors described below. The bed 100 of the embodiment includes a memory that stores information, and a processor that operates based on the information stored in the memory. For example, the information is a program and various types of data. The memory may be the memory 120, or may be another memory. The processor includes hardware. As the processor, various processors including a central processing unit (CPU), a graphics processing unit (GPU), a digital signal processor (DSP), and the like can be used. The memory may be a semiconductor memory such as a static random access memory (SRAM), a dynamic random access memory (DRAM), and a flash memory, may be a register, may be a magnetic storage device such as a hard disk device (HDD), and may be an optical storage device such as an optical disk device. For example, the memory stores a command readable by a computer, and the processor executes the command, thereby implementing a function of the processing unit 110 as processing. The command here may be a command of a command set constituting a program or may be a command that gives an operation instruction to a hardware circuit of the processor.

The memory 120 is a work area of the processing unit 110, and stores various kinds of information. The memory 120 can be implemented by various memories, and the memory may be a semiconductor memory such as SRAM, DRAM, a read only memory (ROM), and a flash memory, may be a register, may be a magnetic storage device, and may be an optical storage device. The memory 120 may store a drive program for driving the driving unit 150 to be described below, and the like.

The communicator 130 is an interface for performing communication via a network, and includes, in a case where the bed 100 performs wireless communication, for example, an antenna, a radio frequency (RF) circuit, and a base band circuit. The bed 100 may perform wired communication, and the communicator 130 in that case may include a communication interface such as a USB connector and an Ethernet connector, a control circuit of the communication interface, and the like. The communicator 130 may operate in accordance with the control by the processing unit 110, or may include a processor for communication control different from the processing unit 110.

The operating unit 140 is an operation interface for controlling the position and angle of the movable part 160. For example, the operating unit 140 may be an operation panel connected to a control box provided with the processing unit 110 in a wired or wireless manner. The operation panel includes, for example, buttons for setting a driving direction and a driving amount of the movable part 160.

The driving unit 150 is an actuator or the like that drives the movable part 160 of the bed 100. The bed 100 of the embodiment may be, for example, a care bed in which the angle of a section, which is a placement surface for the mattress 170, can be adjusted. For example, the bed 100 includes multiple sections sectioned into multiple members as the movable parts 160, and the driving unit 150 adjusts the angles of the sections by changing the position and posture of at least a part of the multiple sections under control of the processing unit 110. The multiple sections include, for example, a back section, an upper leg section, and a lower leg section. For example, by driving of the driving unit 150, control over a back angle (control over the back section) and control over a foot angle (control over the upper leg section and the lower leg section) may be performed.

The bed 100 may be a bed in which the height of the leg unit thereof can be adjusted. In that case, the driving unit 150 is an actuator or the like for driving the leg unit which is the movable part 160. This makes it possible to adjust the total height of the bed 100 by driving of the driving unit 150.

As illustrated in FIG. 2, the bed 100 may be connected to the imaging device 700 via the communicator 130. The processing unit 110 may perform image processing such as object recognition on an image imaged by the imaging device 700. However, the imaging device 700 may be connected to another device such as the server system 300 without via the bed 100.

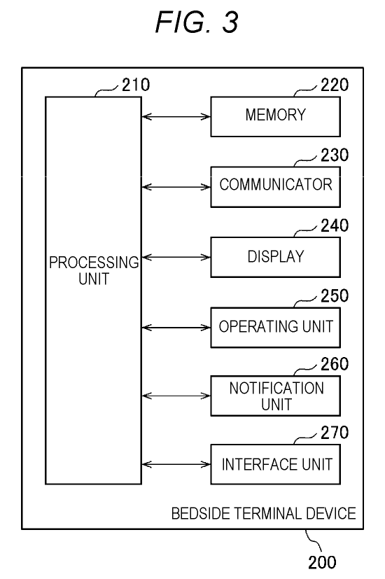

FIG. 3 is a diagram illustrating a configuration example of the bedside terminal device 200. The bedside terminal device 200 includes, for example, a processing unit 210, a memory 220, a communicator 230, a display 240, an operating unit 250, a notification unit 260, and an interface unit 270. As described above, the bedside terminal device 200 may include a display device and a connection device. For example, the display device includes the display 240 in FIG. 3, and the connection device includes the processing unit 210, the memory 220, the communicator 230, the operating unit 250, the notification unit 260, and the interface unit 270 in FIG. 3. However, whether to provide each component illustrated in FIG. 3 at the display device or the connection device can be freely selected, and the components are not precluded from being provided at the display device and the connection device in a distributed manner.

The processing unit 210 is implemented by hardware including at least one of a circuit that processes digital signals and a circuit that processes analog signals. The processing unit 210 may be implemented by processors. As the processors, various types of processors such as CPU, GPU, and DSP can be used. The processor executes a command stored in the memory of the bedside terminal device 200, thereby implementing the function of the processing unit 210 as processing.

The memory 220 is a work area of the processing unit 210, and is implemented by various memories such as SRAM, DRAM, and ROM. For example, the memory 220 stores the vital information acquired from the seat sensor 810 and the measuring device 820, or the like.

The communicator 230 is an interface for performing communication via a network, and includes, for example, an antenna, an RF circuit, and a base band circuit. The communicator 230 performs communication with the server system 300 via a network, for example. The communicator 230 may perform wireless communication or may perform wired communication, and the specific communication method is not limited.

The display 240 is an interface for displaying various types of information, and may be a liquid crystal display, may be an organic EL display, or may be a display using another scheme. The operating unit 250 is an interface for receiving a user operation. The operating unit 250 may be a button or the like that is provided at the display device or the connection device. Moreover, the display 240 and the operating unit 250 may be integrally configured as a touch panel.

The notification unit 260 may include a light emitting unit, a vibration unit, a sound output unit, and the like. The light emitting unit is, for example, a light emitting diode (LED), and makes a notification by light emission. The vibration unit is, for example, a motor, and makes a notification by vibration. The sound output unit is, for example, a speaker, and makes a notification by sound.

The interface unit 270 is an interface used for connecting the measuring device 820 and reading the authentication card 830. The interface unit 270 is, for example, a port that receives connection of a connector such as a universal serial bus (USB). However, as in a case where the authentication card 830 is read using NFC, the communicator 230 may serve as the interface unit 270.

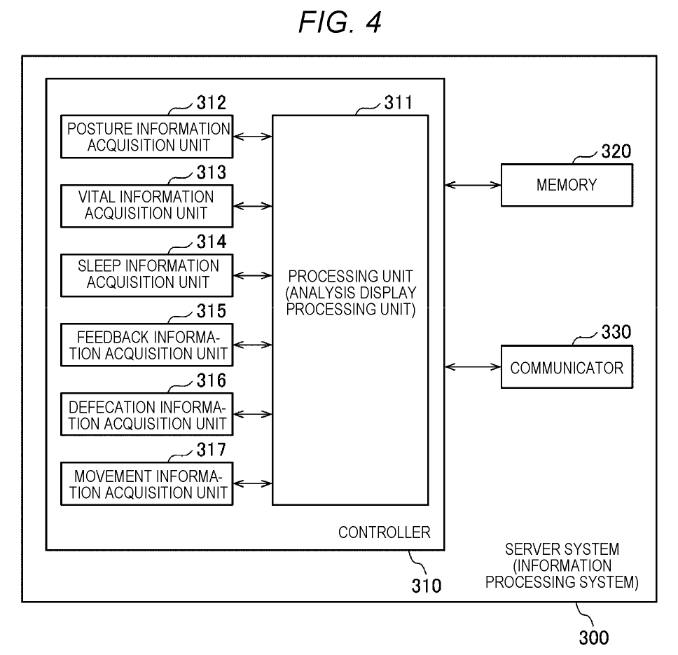

FIG. 4 is a diagram illustrating a detailed configuration example of the server system 300. The server system 300 includes, for example, a controller 310, a memory 320, and a communicator 330.

The controller 310 includes hardware including at least one of a circuit that processes digital signals and a circuit that processes analog signals. The controller 310 may be implemented by processors. As the processors, various types of processors such as CPU, GPU, and DSP can be used. The processor executes a command stored in the memory of the server system 300, thereby implementing the function of the controller 310 as processing.

The controller 310 includes a processing unit 311, a posture information acquisition unit 312, a vital information acquisition unit 313, a sleep information acquisition unit 314, a feedback information acquisition unit 315, a defecation information acquisition unit 316, and a movement information acquisition unit 317. The vital information acquisition unit 313, the sleep information acquisition unit 314, the feedback information acquisition unit 315, and the defecation information acquisition unit 316 in FIG. 4 may be included in a health information acquisition unit (not illustrated). However, the configuration of the controller 310 is not limited to the above. For example, the health information acquisition unit does not necessarily include all the components described above, and some of the vital information acquisition unit 313, the sleep information acquisition unit 314, the feedback information acquisition unit 315, and the defecation information acquisition unit 316 may be omitted. In addition, the movement information acquisition unit 317 can be omitted.

The posture information acquisition unit 312 acquires posture information indicating the posture of the user. For example, as described above, the imaging device 700 may include a posture determination unit, and the posture information acquisition unit 312 may acquire posture information output from the posture determination unit.

The vital information acquisition unit 313 acquires vital information of the user. For example, the vital information may be a respiratory rate, a heartbeat rate, and an activity amount acquired by the seat sensor 810. The vital information acquisition unit 313 may acquire the vital information detected using another sensing device.

The sleep information acquisition unit 314 acquires sleep information indicating the sleep state of the user. For example, the sleep information may be information indicating sleep/awake state or information indicating a sleep depth acquired by the seat sensor 810.

After a predetermined period has elapsed, the feedback information acquisition unit 315 acquires a feedback input by the user in relation to the predetermined period as feedback information. The predetermined period here may be, for example, a period corresponding to one-night sleep. In that case, the feedback information may be information indicating subjective health condition felt by the user when awaking.

The defecation information acquisition unit 316 acquires defecation information about the presence/absence or the number of defecation. The defecation information may be information based on an input by the user, a nurse, or the like, or may be a detection result of a sensing device.

The movement information acquisition unit 317 acquires movement information indicating a control amount of the movable part 160 of the bed 100. In a case where the mattress 170 is an air mattress in which the degree of inflation of each air cell can be controlled, the movement information may include a control amount of the mattress 170.

The processing unit 311 performs analysis processing based on time-series posture information and health information. The health information here is vital information, sleep information, feedback information, defecation information, and the like. Further, the processing unit 311 performs a display process of displaying an analysis processing result to the user or a staff member in charge of the user. That is, the processing unit 311 may be an analysis display processing unit that performs analysis processing and display processing. In addition, the processing unit 311 may perform processing based on the movement information. The processing in each unit of the controller 310 will be described in detail below.

The memory 320 is a work area of the controller 310 and stores various types of information such as posture information, vital information, sleep information, feedback information, defecation information, and movement information. The memory 220 is implemented by various memories.

The communicator 330 is an interface for performing communication via a network, and includes, in a case where the server system 300 performs wireless communication, for example, an antenna, an RF circuit, and a base band circuit. Various modifications can be made to a specific communication method of the communicator 330.

The electronic medical record server 400 is a device including a controller, a memory, a communicator, and the like, similar to the server system 300, for example. A description of the detailed configuration of the electronic medical record server 400 will be omitted.

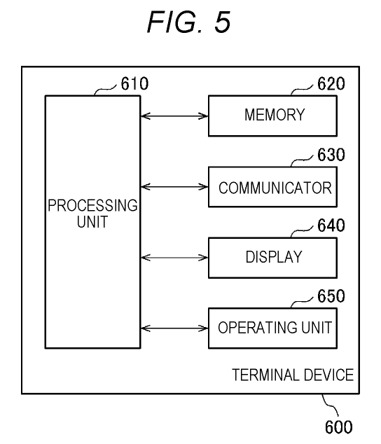

FIG. 5 is a diagram illustrating a detailed configuration example of the terminal device 600. The terminal device 600 may include, for example, a processing unit 610, a memory 620, a communicator 630, a display 640, and an operating unit 650.

The processing unit 610 includes hardware including at least one of a circuit that processes digital signals and a circuit that processes analog signals. The processing unit 610 may be implemented by processors. As the processors, various types of processors such as CPU, GPU, and DSP can be used. The processor executes a command stored in the memory of the terminal device 600, thereby implementing the function of the processing unit 610 as processing.

The memory 620 is a work area of the processing unit 610, and is implemented by various memories such as SRAM, DRAM, and ROM.

The communicator 630 is an interface for performing communication via a network, and includes, for example, an antenna, an RF circuit, and a base band circuit. The communicator 630 performs communication with the server system 300 via a network, for example. For example, the communicator 630 performs communication with the server system 300 via a LAN, and performs communication with the bed 100 and the bedside terminal device 200 using near field communication such as Bluetooth (registered trademark). Various modifications can be made to a specific communication method of the communicator 630.

The display 640 is an interface for displaying various types of information, and may be a liquid crystal display, may be an organic EL display, or may be a display using another scheme. The operating unit 650 is an interface for receiving a user operation. The operating unit 650 may be a button or the like that is provided at the terminal device 600. Moreover, the display 640 and the operating unit 650 may be integrally configured as a touch panel.

The terminal device 600 may include a configuration which is not illustrated in FIG. 5. For example, the terminal device 600 may include various sensors including a motion sensor such as an acceleration sensor and a gyroscope sensor, a pressure sensor, a global positioning system (GPS) sensor, and the like. Moreover, the terminal device 600 may include a light emitting unit, a vibration unit, a sound input unit, a sound output unit, and the like.

The station terminal device 500 is a device including a processing unit, a memory, a communicator, a display, an operating unit, and the like, similar to the terminal device 600, for example. The terminal device 600 may be, for example, a smartphone or a tablet terminal, and the station terminal device 500 may be, for example, a personal computer (PC). A description of the detailed configuration of the station terminal device 500 will be omitted.

As described above, the information processing system 10 according to the embodiment includes a posture information acquisition unit that acquires posture information indicating the posture of a user, a health information acquisition unit that acquires health information indicating the health condition of the user, and a memory. In a case where the information processing system 10 corresponds to the server system 300, the posture information acquisition unit is the posture information acquisition unit 312 in FIG. 4. As described above, the health information acquisition unit includes some or all of the vital information acquisition unit 313, the sleep information acquisition unit 314, the feedback information acquisition unit 315, and the defecation information acquisition unit 316 in FIG. 4. The memory is the memory 320 in FIG. 4. However, as described above, the information processing system 10 may be implemented by a device other than the server system 300, or may be implemented through distributed processing by two or more devices.

The memory 320 stores posture information and health information in association with each other. For example, the posture information acquisition unit 312 acquires posture information in association with a time stamp, and the health information acquisition unit acquires health information in association with a time stamp. The memory 320 may associate the posture information and the health information which are close in timing to each other based on the time stamps.

Then, the processing unit (e.g., the processing unit 311 in FIG. 4) of the information processing system 10 determines the state of the user based on the health information, and executes one or both of the following two processes based on the determination result. The two processes here are (1) a process of obtaining risky posture information based on the posture information associated with the health information in which the state of the user is determined as a given first state, and (2) a process of obtaining safety posture information based on the posture information associated with the health information in which the state of the user is determined as a given second state. The first state indicates a state with a worsening risk, and the second state indicates a state with a low worsening risk.

For example, the health information may be vital information such as a respiratory rate, a heartbeat rate, and an SpO2. Whether the state of the user is the first state is determined based on a value of the vital information. It is known that a normal value range in which there is no problem with the health condition can be set in each vital information. Therefore, the processing unit 311 determines the state based on comparison processing between a value of the vital information and a normal value range associated with the vital information. Specifically, the processing unit 311 may compare a value of the vital information with a normal value range which is a fixed value, may compare degrees of deviations of values of the vital information from the normal value range between postures, or may compare degrees of deviations from the normal value range in time-series vital information, or these processing may be implemented by machine learning. For example, the processing unit 311 may determine the first state when a value of the vital information deviates from the normal value range which is a fixed value. Alternatively, the processing unit 311 may obtain a statistic value of the vital information for each posture, and when the vital information in which the degree of a deviation from a normal range is larger than other statistic values is obtained, may determine that the user is in the first state. Alternatively, the processing unit 311 may perform intercomparison of time-series vital values of the same user, and when the vital information in which the degree of a deviation from a normal range is larger than other values is obtained, may determine that the user is in the first state. Similarly, in the determination of the second state, the processing unit 311 may compare a value of the vital information and a normal value range which is a fixed value, may compare degrees of matching between values of the vital information and the normal value range between postures, or may compare degrees of matching with the normal value range in time-series vital information. The processing unit 311 determines the second state when a value of the vital information falls within a fixed normal value range, or when the degree of matching between a value of the vital information and the normal value range is relatively high. The processing will be described in detail below. Similarly, in a case where health information other than vital information is used, for example, a normal range is set for each health information, and the processing unit 311 compares acquired health information itself or information obtained from the health information with the normal range, thereby executing at least one of determination on whether the user is in the first state or determination on whether the user is in the second state.

The risky posture means a posture in which a risk of worsening the state of the user is relatively high. The safety posture means a posture in which a risk of worsening the state of the user is relatively low. A process of obtaining the risky posture information or the safety posture information based on the posture information may be a process of setting the posture information itself associated with the health information as the risky posture information or the safety posture information. Alternatively, a process of obtaining the risky posture information or the safety posture information based on the posture information may be a process of obtaining a posture range or an average posture based on multiple pieces of posture information respectively associated with multiple pieces of health information.

Although there are differences between individual users as to what posture has what influence on the health condition, in the method of the embodiment, it is possible to specify a risky posture and a safety posture for a user while confirming the actual health information of the user. Therefore, the risky posture or the safety posture or the both of the user can be obtained with accuracy. As a result, subsequent nursing-care/care using the risky posture and the safety posture can be appropriately executed.

A part or all of the processing that is performed by the information processing system 10 of the embodiment may be implemented by a program. The processing that is performed by the information processing system 10 is processing performed by the server system 300 in a narrow sense, but may include processing executed by other devices such as the terminal device 600 and the bedside terminal device 200.

The program according to the embodiment can be stored, for example, in a non-transitory information storage medium (information storage device) which is a computer-readable medium. The information storage medium can be implemented by an optical disc, a memory card, an HDD, or a semiconductor memory, for example. The semiconductor memory is a ROM, for example. The controller 310 and the like perform the various types of processing in the embodiment based on the program stored in the information storage medium. That is, the information storage medium stores the program for causing a computer to function as the controller 310 and the like. The computer is a device including an input device, a processing unit, a memory, and an output unit. Specifically, the program according to the embodiment is a program for causing the computer to execute respective steps, which will be described below using FIGS. 6, 10, 15 to 17, 21 to 22, and the like.

The method of the embodiment can be applied to an information processing method by an information processing system. The information processing method includes, by the information processing system, acquiring posture information indicating a posture of a user, acquiring health information indicating a health condition of the user, storing the posture information and the health information in association with each other, determining a state of the user based on the health information, and performing at least one of (1) a process of obtaining risky posture information based on the posture information associated with the health information in which the state of the user is determined as a first state with a worsening risk, and (2) a process of obtaining safety posture information based on the posture information associated with the health information in which the state of the user is determined as a second state with a relatively-low worsening risk.

### 2. Posture Determination Processing

A flow of processing using posture information and health information will be described. Hereinafter, processing related to a risky posture, a safety posture, and a specific risky posture will be separately described. The specific risky posture will be described in detail below. In addition, an example in which a representative posture specified by posture information during a predetermined period will also be described. Note that a description will be made below on an example in which vital information is used as health information in the processing related to a risky posture, a safety posture, and a specific risky posture, and sleep information, excretion information, and feedback information are used as health information in the processing related to a representative posture. However, each health information may be replaced with another health information.

### 2.1 Risky Posture

FIG. 6 is a flowchart illustrating a process of obtaining risky posture information indicating a risky posture. In step S101, the posture information acquisition unit 312 acquires posture information indicating the posture of a user. The term "acquisition" here may be a process of receiving posture information, which is obtained by another device, via a communication interface (the communicator 330). Alternatively, "acquisition" may be a process of obtaining posture information based on sensing data acquired by another device. The device used to acquire the posture information is the imaging device 700, for example.

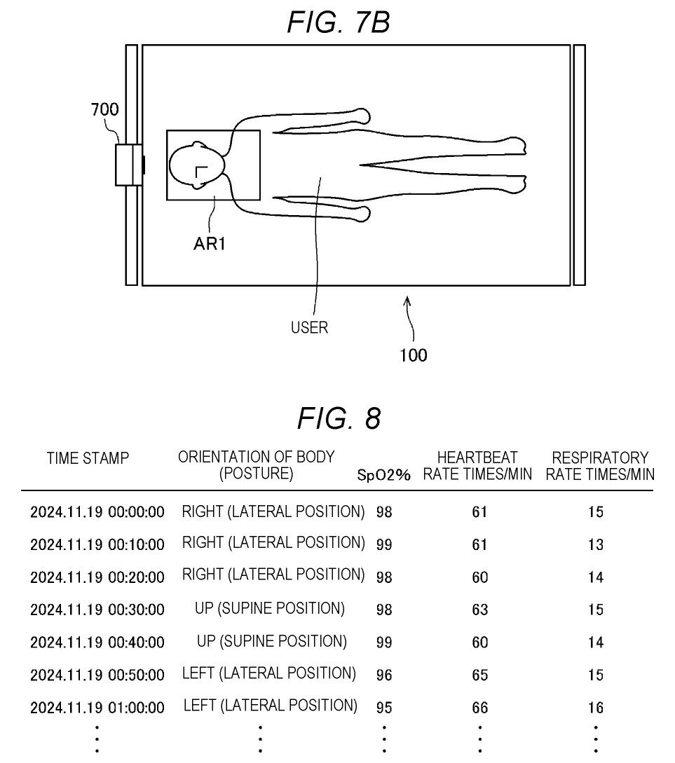

FIGS. 7A and 7B are diagrams each illustrating an example of a positional relationship between the bed 100 and the imaging device 700. As illustrated in FIGS. 7A and 7B, the imaging device 700 may be disposed at a head board of the bed 100. The imaging device 700 images an imaging range AR1 corresponding to at least the face portion of the user lying down on the bed 100. The imaging device 700 outputs an imaged image including the face region of the user. Here, the imaging device 700 may be an infrared camera (an infrared sensor). The imaging range of the imaging device 700 is not limited to the imaging range AR1 illustrated in FIGS. 7A and 7B. For example, the imaging device 700 may be capable of imaging a region other than the face of the user.

A posture indicated by the posture information may be any one of multiple postures including at least one of a supine position, a right lateral position, a left lateral position, and a prone position. For example, a method of determining a sleeping posture using an infrared sensor is described in Japanese Patent Application No. 2022-120652, filed on Jul. 28, 2022, entitled "SLEEPING POSTURE DETECTION SYSTEM, SLEEPING POSTURE MANAGEMENT SYSTEM, AND ENVIRONMENTAL MANAGEMENT SYSTEM". This patent application is incorporated herein in its entirety by reference. In the embodiment, the posture information is acquired using this method.

However, the sensor used to acquire the posture information is not limited to the imaging device 700 (including an infrared sensor). For example, a motion sensor may be directly attached to the user. The posture information acquisition unit 312 obtains the posture information based on an output of the motion sensor. The motion sensor here may be an acceleration sensor or may be an angular velocity sensor. For example, the motion sensor is a six-axis motion sensor that detects accelerations in directions of three axes and angular velocities around the respective axes.

The form of the posture information is also not limited to the above example. For example, the posture information may be information of the posture of the user on the bed 100, including a rotation angle in the left-right direction of the user with respect to a given reference and a raise-up angle of the position of the head or the feet with respect to a reference position. For example, in a case where an angle at a supine position is set as a reference angle (0°), a rotation angle in the left-right direction may indicate a rotation amount with respect to the reference angle. The raise-up angle of the head or the feet may indicate an angle indicating a degree of raise-up in a case where an angle indicating a posture parallel to a horizontal plane is set as a reference angle (0°).

For a process of obtaining a risky posture, an example in which the posture information indicates any one of a supine position, a right lateral position, a left lateral position, and a prone position will be described below. In addition, for a process of obtaining a safety posture to be described below, an example in which the posture information indicates a rotation angle in the left-right direction and a raise-up angle will be described. However, in the process of obtaining a risky posture, the posture information indicating a rotation angle in the left-right direction and a raise-up angle may be used. In addition, in the process of obtaining a safety posture, the posture information indicating any one of a supine position, a right lateral position, a left lateral position, and a prone position may be used. Further, the posture information according to the embodiment is only required to be information capable of specifying the posture of the user, and information other than those in the above examples may be used. Furthermore, in other processing such as a process related to a specific risky posture and a process of obtaining a representative posture, various types of information can be used as the posture information in a similar manner. In other words, even when posture information is specifically exemplified in the present specification, such posture information can be replaced with another form of posture information.

In step S101 in FIG. 6, the posture information acquisition unit 312 periodically executes a process of obtaining posture information at predetermined intervals. For example, the imaging device 700 acquires time-series imaged images by performing imaging in accordance with a given frame rate, and outputs time-series posture information by repeatedly performing a process of obtaining posture information based on the imaged images. The posture information acquisition unit 312 acquires posture information and time information indicating an acquisition timing of the posture information in association with each other. The time information is, for example, a time stamp including year, month, date, hour, minute, and second. Note that the acquisition timing here is an imaging timing of an imaged image used for calculating posture information in a narrow sense, but other information such as a calculation timing of the posture information may be used.

In step S102, the vital information acquisition unit 313 acquires vital information. The vital information acquisition unit 313 may receive vital information acquired by another device, or may perform a process of obtaining vital information based on sensing data detected by another device.

The vital information may include information of a respiratory rate and a heartbeat rate. For example, the seat sensor 810 illustrated in FIG. 1 acquires a sensor value of the load sensor at a given rate and periodically executes calculation processing based on the sensor value, thereby calculating time-series respiratory rate and heartbeat rate. The vital information acquisition unit 313 acquires information indicating a respiratory rate and a heartbeat rate directly or via another device such as the bedside terminal device 200 from the seat sensor 810.

The vital information may include information of SpO2, a blood pressure, a body temperature, and the like. In that case, the device used for acquiring the vital information may be a device attached to a fingertip, may be a wristwatch-type device, or may be another form of device. The vital information acquisition unit 313 acquires the vital information based on outputs of these devices.

The vital information acquisition unit 313 acquires vital information and time information indicating an acquisition timing of the vital information in association with each other. The time information is, for example, a time stamp. The acquisition timing is a detection timing of sensing data used for calculating vital information in a narrow sense, but other information such as a calculation timing of vital information may be used.

In step S103, the controller 310 causes the memory 320 to store the posture information and the vital information in association with each other. For example, the controller 310 compares a time stamp associated with the posture information and a time stamp associated with the vital information, thereby performing a process of associating the posture information and the vital information a difference in acquisition timing between which is equal to or smaller than a predetermined threshold value.

FIG. 8 is a diagram illustrating an example of data stored in the memory 320. As illustrated in FIG. 8, the memory 320 stores data in which time information, posture information, and vital information are associated with each other. The time information is a time stamp. The posture information is information that specifies any one of a supine position, a right lateral position, a left lateral position, and a prone position. The vital information includes SpO2, a heartbeat rate, and a respiratory rate.

In the example of FIG. 8, at 0:00:00 on Nov. 19, 2024, the posture was a right lateral position, and the vitals at that time were such that the SpO2 was 98%, the heartbeat rate was 61 times/minute, and the respiratory rate was 15 times/minute. Similarly at other timings, the memory 320 stores information in which the state of the vitals of the user is associated with each posture. Note that in FIG. 8, an example in which SpO2, a heartbeat rate, and a respiratory rate are associated with each posture has been described, but this is not a limitation. For example, depending on a relationship between an acquisition rate of posture information and an acquisition rate of vital information, one piece of posture information may be associated with multiple SpO2, multiple pieces of posture information may be associated with one SpO2, or multiple pieces of posture information may be associated with multiple SpO2. The same applies to association between posture information and heartbeat rate, and association between posture information and respiratory rate.

In the flowchart in FIG. 6, the information processing system 10 may repeatedly execute processing in steps S101 to S103. For example, in a case where data for one week are accumulated, the information processing system 10 may execute processing using the data for one week. In other words, in the flowchart in FIG. 6, the processing in and after step S104 may be executed on condition that the processing in steps S101 to S103 has been repeated for a predetermined period.

In step S104, the processing unit 311 (analysis display processing unit) performs analysis processing on accumulated posture information and health information (e.g., vital information). For example, the processing unit 311 obtains multiple statistic values, which are statistic values of the vital information, for each of multiple postures (a supine position, a right lateral position, a left lateral position, and a prone position), and performs an analysis process of obtaining risky posture information based on comparison processing on the multiple statistic values.

In the processing in step S104, the processing unit 311 first performs a process of classifying the data indicated in FIG. 8 in accordance with posture information. Specifically, the processing unit 311 classifies the data into a first data group in which the posture information indicates a supine position, a second data group in which the posture information indicates a right lateral position, a third data group in which the posture information indicates a left lateral position, and a fourth data group in which the posture information indicates a prone position.

Then, the processing unit 311 performs statistical processing on each data group. For example, the processing unit 311 performs a process of obtaining an average value, a maximum value, and a minimum value of multiple SpO2 values included in the first data group. Further, the processing unit 311 obtains an average value, a maximum value, and a minimum value of multiple respiratory rates included in the first data group, and an average value, a maximum value, and a minimum value of multiple heartbeat rates included in the first data group. As a result, the statistic values of the vital information corresponding to the supine position can be obtained. The processing unit 311 performs the same processing on the second data group to the fourth data group, thereby obtaining the statistic values of the vital information corresponding to the right lateral position, the statistic values of the vital information corresponding to the left lateral position, and the statistic values of the vital information corresponding to the prone position.

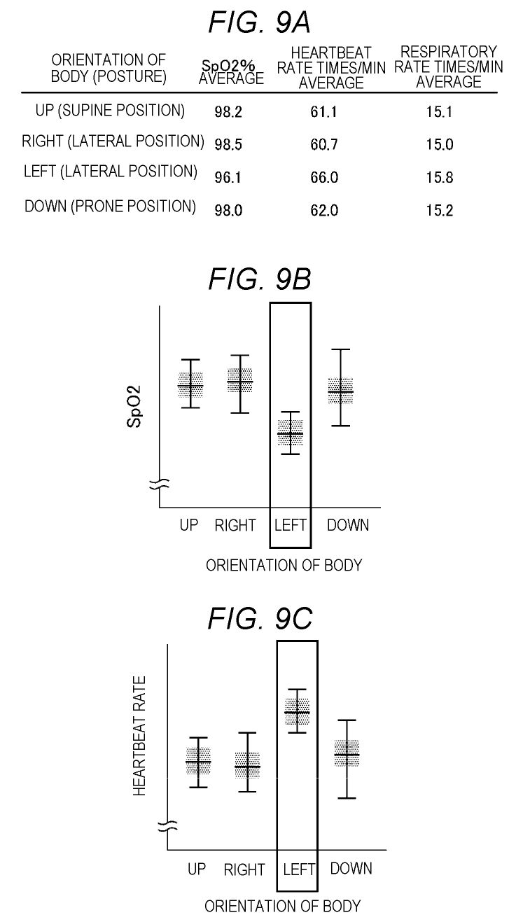

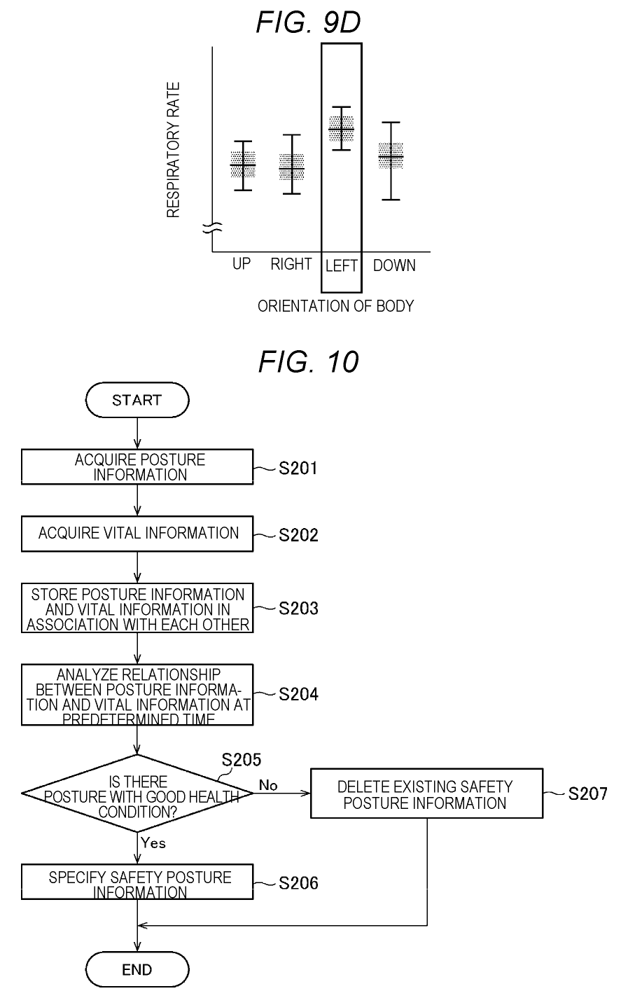

FIG. 9A is a diagram illustrating an example of statistic values obtained for each posture. Note that in FIG. 9A, average values are exemplified as statistic values, but maximum values or minimum values may be obtained as described above. In addition, the statistic values here may include other information such as median values, dispersions, and standard deviations. Further, the average values are not limited to simple average, and may include other information based on the average values (e.g., trimmed average).

Each of FIGS. 9B to 9D is a diagram illustrating statistic values of SpO2 for each posture, statistic values of heartbeat rate for each posture, and statistic values of respiratory rate for each posture. The horizontal axis in FIGS. 9B to 9D represents posture, where "Up" indicates the supine position, "Right" indicates the right lateral position, "Left" indicates the left lateral position, and "Down" indicates the prone position. The vertical axis represents each vital information value. In the vertical axis in FIGS. 9B to 9D, boxplots each indicating a minimum value, a first quartile, a median value, a third quartile, and a maximum value of the vital information are exemplified.

In step S105 in FIG. 6, the processing unit 311 determines whether there is a posture in which the health condition is bad. For example, the processing unit 311 compares the statistic values of the vital information between multiple postures, thereby determining a posture in which the user is in the first state. Specifically, the processing unit 311 determines a posture in which the health condition is relatively bad as compared with other postures.

For example, SpO2 is preferably closer to 100%, and lowering of the value thereof indicates a possibility that the health condition is worsened. Therefore, in a case where there is a posture in which the statistic value of SpO2 is relatively low as compared with other postures, the processing unit 311 may determine the posture as a risky posture. In the examples in FIGS. 9A and 9B, the statistic value of SpO2 in the left lateral position is apparently lower than the statistic values of SpO2 in other postures (the supine position, the right lateral position, and the prone position). For example, in a case where the statistic value of SpO2 in a given posture is lower by a predetermined amount or more or by a predetermined ratio or more than the statistic values of SpO2 in other postures, the processing unit 311 determines that the health condition is worsened in the given posture.

In a case where it is determined that there is a posture in which the health condition is bad (step S105: Yes), in step S106, the processing unit 311 determines the posture as a risky posture. In the examples in FIGS. 9A and 9B, the processing unit 311 determines the left lateral position as a risky posture in which a risk of worsening the health condition of the user is present. In other words, the processing unit 311 determines posture information associated with vital information indicating worsening of the health condition (posture information indicating the left lateral position in the above examples) as risky posture information indicating a risky posture.

In the determination on a posture with bad health condition, vital information other than SpO2 may be used. For example, it is known that, for the heartbeat rate and the respiratory rate, standard ranges of values can be set for each user, and the values deviate from the standard ranges (exceed upper limits in a narrow sense) as the health condition is worsened. Therefore, in a case where there is a posture in which the statistic value of heartbeat rate is relatively high as compared with other postures, the processing unit 311 may determine the posture as a risky posture. Similarly, in a case where there is a posture in which the statistic value of respiratory rate is relatively high as compared with other postures, the processing unit 311 may determine the posture as a risky posture. In the examples in FIGS. 9A, 9C, and 9D, the statistic values of heartbeat rate and respiratory rate in the left lateral position are apparently higher than the statistic values of heartbeat rate and respiratory rate in other postures (the supine position, the right lateral position, and the prone position). Therefore, the processing unit 311 determines that there is a posture in which the health condition is bad (step S105: Yes), and determines the left lateral position as a risky posture (step S106).

In a case where risky posture information is determined, the memory 320 stores the risky posture information in association with the user. In the examples in FIGS. 9A to 9D, posture information corresponding to the left lateral position is stored as the risky posture information in association with the user subjected to the processing. By storing the risky posture information, it is possible to reduce health risks of the user due to postures. For example, the information processing system 10 executes processing in which the user avoids a risky posture. Specific processing using the risky posture information will be described below using FIGS. 19 to 22.

In a case where there is no posture in which the health condition is bad (step S105: No), the target user shows no worsening of the health condition in any posture. For example, even in a case where risky posture information is specified and stored in steps S105 to S106 executed in the past, when no risky posture information is specified in subsequent steps S105 to S106, it is conceivable that a risk of worsening the health condition of the user in the stored risky posture information has been sufficiently lowered. Thus, in a case where there is no posture in which the health condition is bad (step S105: No), in step S107, the processing unit 311 may perform a process of deleting the stored risky posture information. Accordingly, in a case where a risk of worsening the health condition due to a posture is lowered, the risky posture information can be automatically updated (deleted).

In the foregoing, a description has been made on a process of determining a posture in which the health condition is relatively bad by comparing vital information between multiple postures. However, the processing of the embodiment is not limited thereto. For example, in step S105 in FIG. 6, the processing unit 311 may determine whether there is a posture in which the health condition is bad by comparing statistic values of vital information obtained for each posture with a given threshold value. The threshold value here may be a fixed value or may be a value dynamically determined based on the gender, age, body height, weight, and the like of the user.

Alternatively, the processing unit 311 may determine whether there is a posture in which the health condition is bad by comparing statistic values of vital information obtained in the past for the same user with statistic values of the latest vital information of the user for each posture. For example, in a case where the values of the latest vital information have been changed in a worsening direction by a predetermined amount (or a predetermined ratio) or more from the values of the past vital information, the processing unit 311 determines that the health condition is bad. In addition to the above, various modifications can be made to the process of determining whether the health condition is bad based on statistic values of vital information.

As will be described below using FIG. 12, the health condition may be determined based on time-series changes in vital information, and a process of obtaining statistic values of the vital information is not essential. In addition, the risky posture in the embodiment is not limited to a posture in which the health condition is bad, and may include all postures in which the health condition is not good. For example, the processing unit 311 may distinguish between a posture with a bad health condition, a posture with a good health condition, and a posture with a medium health condition based on vital information. In that case, the risky posture may include both a posture with a bad health condition and a posture with a medium health condition. Further, the processing unit 311 may determine, based on vital information, only a posture with a good health condition, and may determine any other postures as risky postures.

### 2.2 Safety Posture

FIG. 10 is a flowchart illustrating a process of obtaining safety posture information indicating a safety posture. In step S201, the posture information acquisition unit 312 acquires posture information indicating a posture of the user. In step S202, the vital information acquisition unit 313 acquires vital information. In step S203, the controller 310 causes the memory 320 to store the posture information and the vital information in association with each other.

The processing in steps S201 to S203 is the same as the processing in steps S101 to S103 in FIG. 6. That is, the controller 310 acquires time-series posture information and time-series vital information, and causes the memory 320 to store the posture information and the vital information in a state of being associated with each other based on time stamps. As the posture information and the vital information, various types of information can be used as described above. In addition, in a case where a process of obtaining both of a risky posture and a safety posture is performed, the processing in steps S101 to S103 and the processing in steps S201 to S203 may be commonalized.

In a case where data for a predetermined period are accumulated, in step S204, the processing unit 311 (analysis display processing unit) performs analysis processing on the accumulated posture information and vital information. The specific processing may be the same as those in the examples described above using FIGS. 9A to 9D, for example.

In step S205, the processing unit 311 determines whether there is a posture in which the health condition is good. For example, the processing unit 311 compares the statistic values of the vital information between multiple postures, thereby determining a posture in which the health condition is relatively good as compared with other postures (the user is in the second state).

The posture in which the health condition is good here may include all postures in which the health condition is not bad. For example, in the examples illustrated in FIGS. 9A to 9D, the processing unit 311 determines all of the supine position, the right lateral position, and the prone position other than the left lateral position, which is determined as bad health condition, as postures in which the health condition is good. Alternatively, the multiple postures are not limited to a posture in which the health condition is good and a posture in which the health condition is bad, and may include a posture in which the health condition is medium. For example, in the examples illustrated in FIGS. 9A to 9D, the processing unit 311 determines only a part of the supine position, the right lateral position, and the prone position as a posture in which the health condition is good. Specifically, the processing unit 311 determines a part of postures in which values of vital information are close to ideal values (one posture in a narrow sense) as a posture in which the health condition is good. Similarly, in a case where vital information is compared with a given threshold value or compared with a past history of the same user, the number of postures in which the health condition is good may be one or more, and specific determination processing can be modified in various ways.

In a case where it is determined that there is a posture in which the health condition is good (step S205: Yes), in step S206, the processing unit 311 determines the posture as a safety posture. In other words, the processing unit 311 determines posture information associated with vital information indicating improvement in the health condition as safety posture information indicating a safety posture. The processing unit 311 causes the memory 320 to store the safety posture information. By storing the safety posture information, it is possible to reduce health risks of the user due to postures. For example, the information processing system 10 executes processing that enables the user to actively take a safety posture. Specific processing using the safety posture information will be described below using FIGS. 19 to 22.

In a case where there is no posture in which the health condition is good (step S205: No), in step S207, the processing unit 311 performs a process of deleting the stored safety posture information.

The above-described process of obtaining a safety posture may be applied to a case subjected to postural drainage which is a care for the user to remove phlegm accumulated in lungs by adjusting the posture. In that case, the health information acquisition unit (the vital information acquisition unit 313) acquires vital information, which is a sensing result related to a lung activity of the user, as health information. The processing unit 311 determines the state of the lungs of the user based on the vital information, and performs a process of obtaining safety posture information indicating a posture suitable for the postural drainage of the user based on posture information associated with the vital information in which the state of the lungs is determined as a third state corresponding to a good state. The third state here is a state in which the degree of matching between a value of the vital information and a normal value range is a predetermined value or greater, and is a state included in the first state described above. In the following, an example in which a range indicating a safety posture is obtained as safety posture information will be described, but the safety posture information may be one given posture.

A care for removing phlegm accumulated in lungs by adjusting a body position (posture) and utilizing the gravity is referred to as "postural drainage". At which part of the lungs phlegm is likely to accumulate varies from user to user, and the posture suitable for the postural drainage depends on the part. In the related art, specialized skills such as palpation, auscultation, visual inspection, and the like have been required to determine a site where phlegm is accumulated in the lungs. In addition, even when a site where phlegm is accumulated is known, specialized knowledge is required to determine a posture suitable for the site. In this respect, in the method of the embodiment, it is possible to determine whether the posture is suitable for the postural drainage based on vital information. Therefore, even when a site where phlegm is accumulated cannot be determined, and/or without specialized knowledge, it is possible to appropriately determine a posture suitable for postural drainage. Hereinafter, specific processing in a case of postural drainage will be described.

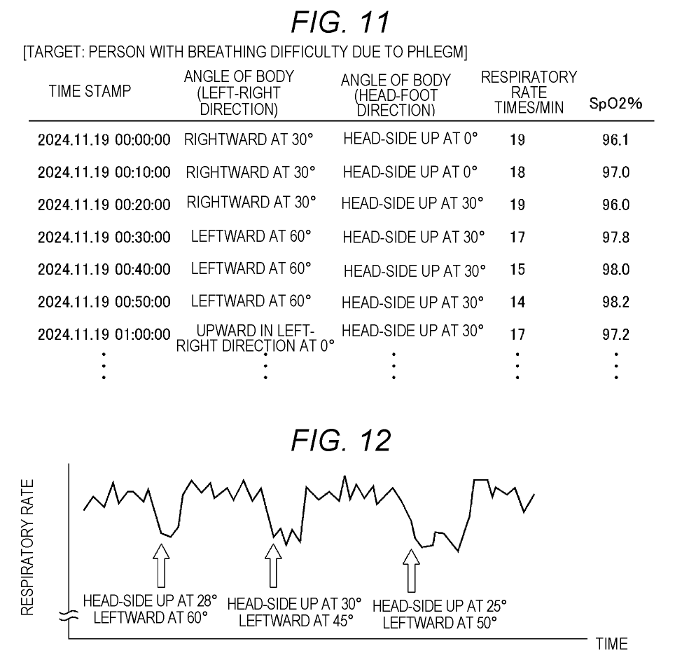

FIG. 11 illustrates an example of data stored in the memory 320 by the processing in steps S201 to S203. As illustrated in FIG. 11, the memory 320 stores data in which time information, posture information, and vital information are associated with each other. The time information is a time stamp. The posture information is a combination of a rotation angle in the left-right direction and a raise-up angles of the head and feet. The vital information indicating a lung activity is, for example, a respiratory rate and SpO2. However, as the vital information, other information related to the lungs (respiration) may be used.

FIG. 12 is a graph showing time series changes in vital information (respiratory rate) for a predetermined period. In FIG. 12, the horizontal axis represents time and the vertical axis represents respiratory rate. In a case where the state of the lungs is good, the respiratory rate relatively decreases because oxygen can be efficiently taken in. Therefore, the processing unit 311 may determine a timing at which the respiratory rate decreases based on the time-series changes in the vital information, and may obtain the posture information corresponding the timing as the safety posture information. The decrease in respiratory rate here may be the respiratory rate falling below a predetermined threshold value, or may be the respiratory rate being relatively lower than the respiratory rates at other timings. Alternatively, the processing unit 311 may perform a process of comparing a change amount of the vital information with a threshold value. In addition, the health condition may be determined by performing a signal processing method such as Fourier transform or filtering on the time-series changes in the vital information. Further, the processing unit 311 may perform determination on the health condition based on the vital information using a machine learning method such as auto encoder. Alternatively, a medical doctor or a nurse may actually input information related to the state of a patient who is a monitoring target, and the health condition may be determined by a model using a supervised learning method in which the information is used as a label, and various modifications can be made to the specific processing.

In the example in FIG. 12, the respiratory rate decreases at three timings indicated by arrows, and thus it is determined that the state of the lungs is good. The posture information corresponding to the respective timings are "leftward at 60°, head-side up at 28°", "leftward at 45°, head-side up at 30°", and "leftward at 50°, head-side up at 25°". The processing unit 311 determines safety posture information based on these pieces of posture information.

For example, the processing unit 311 may set an aggregation of these multiple pieces of posture information as safety posture information. Alternatively, the processing unit 311 may obtain information indicating a posture range specified by multiple pieces of posture information as safety posture information. In the example in FIG. 12, the processing unit 311 sets a range of "leftward at 45 to 60°, head-side up at 25 to 30°" including the three postures as safety posture information. Alternatively, the processing unit 311 may obtain average values of rotation angles in the left-right direction and raise-up angles, and may set a posture falling within a predetermined range including the average values as a safety posture.

In addition, a case is also conceivable in which the respiratory rate tends to decrease when the posture changes in the order of the left lateral position, the supine position, and the left lateral position, but the respiratory rate tends not to decrease when the left lateral position is simply continued. In this case, it is conceivable that a posture change pattern of the left lateral position, the supine position, and the left lateral position is suitable for improvement in the state of the lungs. Therefore, the processing unit 311 may set a posture pattern indicating time-series changes of multiple postures as safety posture information.

The processing unit 311 may perform a control of driving a movable part of the bed 100 or the mattress 170 used by the user or movable parts of the both in a predetermined range in a target period of acquisition of vital information and posture information. The movable part of the bed 100 is, for example, multiple sections of the bed 100. The movable part of the mattress 170 (air mattress) may be multiple air cells each of which can be changed in thickness.

As described above, in the method of the embodiment, the processing in steps S201 to S203 is continued for a predetermined period, and then a process of searching for a posture by which the state of the lungs is improved, by performing analysis processing based on posture information and vital information. Therefore, in a case where there is little change in the posture of the user during a target period, the user has not taken a posture suitable for postural drainage. For example, when the posture is fixed due to the user's preference or the like, a posture by which the state of the lungs is improved may not be detected during a predetermined period. In particular, in considering raising up of the head or feet as posture information, a posture that is not easy to take without an external factor, such as control of the bed 100 or the mattress 170 or arrangement of a cushion, is also conceivable. Similarly, there is a possibility that a safety posture does not appear when there is little change in the posture, also in cases other than postural drainage.

In this respect, it is possible to cause the user to take a variety of postures by changing the movable parts of the bed 100 and the mattress 170 within certain ranges. When vital information about a variety of postures is acquired, a posture by which the health condition is improved is more likely to appear, and thus a safety posture can be more easily specified.

Note that the movable amount of the movable part of the bed 100 corresponds to change amounts of the angle and height of each section, and is controlled by the driving amount of the driving unit (a driving motor or the like). In addition, the movable amount of the movable part of the mattress 170 corresponds to the thickness of each air cell, and is controlled by the driving amount (pressure value) of a pump that supplies gas to the air cells. Therefore, controlling the movable part in a predetermined range may be driving the driving motor of the bed 100 in a predetermined range, or may be changing the pressure of the pump of the mattress 170 in a predetermined range. A specific driving range can be set as appropriate in accordance with a type of a posture that the user is desired to take.

### 2.3 Specific Risky Posture

In the foregoing, a description has been made on a process of obtaining a risky posture or a safety posture based on posture information and vital information in a situation where it is unknown what the risky posture or the safety posture is like. However, a case in which a posture that can be risky for the health of a certain user is known is conceivable.

For example, in the case of a pregnant female in a late gestation (e.g., 28 to 40 weeks), it is known that sleeping in a supine position increases a risk of supine hypotension syndrome in which the blood pressure is lowered, and tachycardia, vomiting, cold sweat, dizziness, and the like are caused. That is, the supine position tends to be highly risky for a user who is a pregnant female. However, for users who are pregnant females, the degree of a risk due to the supine position varies from user to user. Further, even in a case where a risk of the supine position is high, the number of weeks of pregnancy from which users should be cautious about the risk varies from user to user. Since a case where the supine position is a comfortable posture for a certain user is also conceivable, it is not preferable to uniformly prohibit the supine position even for a pregnant female. Thus, the information processing system 10 may perform a process of evaluating such a risk related to a posture.

The memory 320 stores specific risky posture information indicating a specific risky posture which is a posture that may worsen the state of the user. The specific risky posture information may be stored in association with an attribute of the target user. In the above example, information indicating the supine position is stored in the memory 320 as the specific risky posture information in association with a user attribute of "pregnant female" or "late gestation".

The processing unit 311 determines, based on posture information and specific risky posture information, whether the posture of the user indicated by the posture information is a specific risky posture.

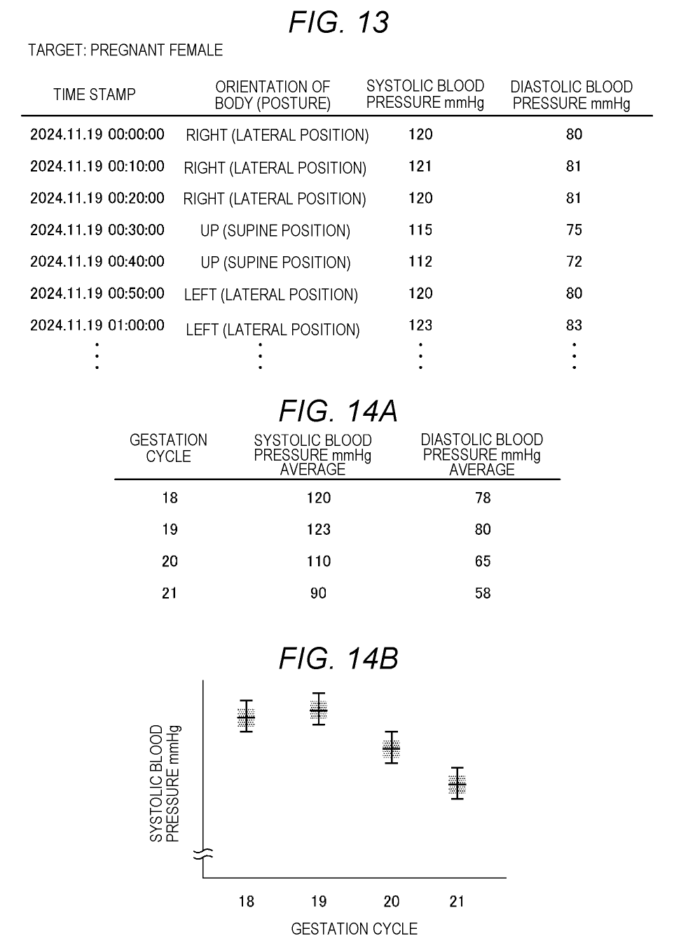

FIG. 13 is a diagram illustrating an example of data acquired from a target user who is a pregnant female. As illustrated in FIG. 13, the memory 320 stores data in which time information, posture information, and vital information are associated with each other. The time information is a time stamp. The posture information indicates any one of a supine position, a right lateral position, a left lateral position, and a prone position. The vital information is information about the supine hypotension syndrome, and specifically may be systolic blood pressure and diastolic blood pressure values.

The processing unit 311 performs a process of extracting a record including posture information corresponding to specific risky posture information from the data illustrated in FIG. 13. In an example of the supine hypotension syndrome, the processing unit 311 extracts vital information associated with posture information indicating a supine position.

Then, the processing unit 311 determines whether the state of the user has been worsened based on the vital information associated with the posture information determined as a specific risky posture, thereby determining (evaluating) the risk of the specific risky posture for the user who is a processing target. Determination of a risk may be determination on presence or absence of the risk, or may be determination on the degree of the risk.

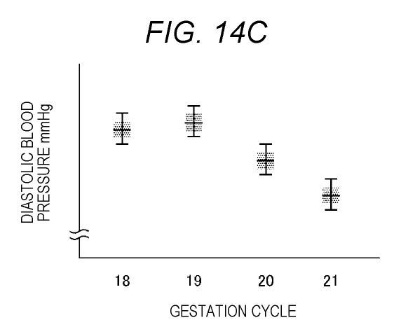

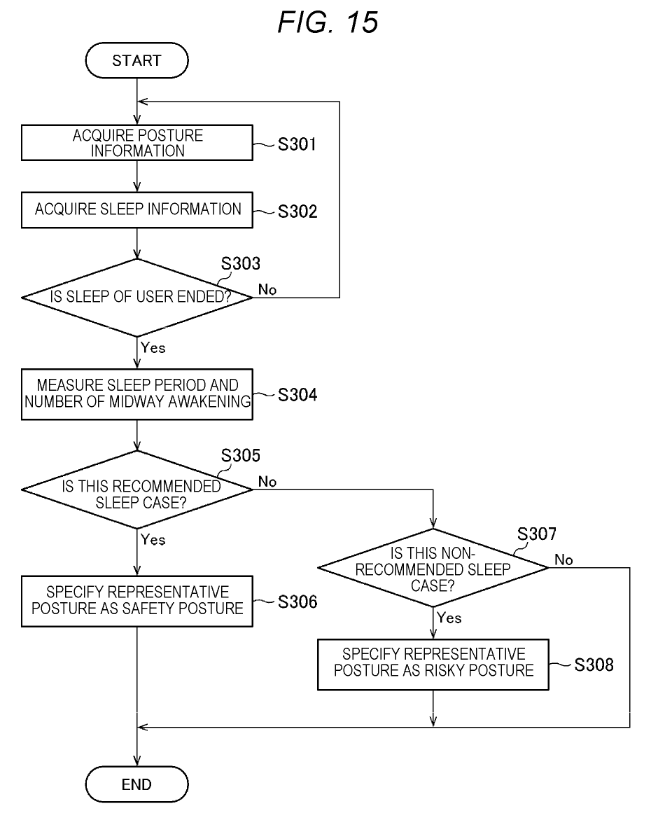

For example, the processing unit 311 may obtain statistic values of vital information as in the example of obtaining risky posture information. FIG. 14A is a diagram illustrating an example of statistic values of blood pressure values in a supine position according to a gestation cycle. FIG. 14B is a diagram illustrating a relationship between gestation cycle and systolic blood pressure, and FIG. 14C is a diagram illustrating a relationship between gestation cycle and diastolic blood pressure. The horizontal axes of FIGS. 14B and 14C represent gestation cycle (week) and the vertical axes represent blood pressure value. In FIGS. 14B and 14C, box charts are exemplified on the vertical axes as in FIGS. 9B to 9D.

For example, the processing unit 311 may determine whether the blood pressure value is lowered by a predetermined value or more based on time-series changes in the blood pressure value (changes in statistic values of the blood pressure values per week) of the target patient. In the examples in FIGS. 14A to 14C, at a stage of 21 weeks of pregnancy, both the systolic blood pressure and the diastolic blood pressure decrease to a recognizable degree as compared before 20 weeks. For example, the processing unit 311 obtains blood pressure values for four weeks and, in a case where the values in the fourth week decrease by a predetermined ratio (20%) or more with reference to those in the first week, determines that there is a risk of supine hypotension syndrome for a target patient. In addition, the processing unit 311 may determine whether the risk is high or low in accordance with the degree of decrease in the blood pressure values.

In this way, according to the method of the embodiment, in a case where a risk depending on a specific posture is expected, it is possible to automatically determine the presence or absence of the risk at an early stage. Specifically, according to the method of the embodiment, in determining a risk based on vital information, only the vital information associated with a specific risky posture is subjected to the processing, so that it is possible to prevent data of other postures from contributing to a result as noises.

### 2.4 Processing Based on Representative Posture

In the foregoing, processing based on data in which posture information and vital information are associated with each other has been described. However, the method of the embodiment is not limited thereto. For example, a representative posture which is a representative posture in a predetermined period may be obtained from posture information, and the representative posture may be set as a risky posture or a safety posture. Some processes using a representative posture will be described below.

### 2.4.1 Sleep Case

The health information acquisition unit (sleep information acquisition unit 314) of the information processing system 10 according to the embodiment may acquire sleep information indicating the sleeping state of the user as health information. Then, the processing unit 311 may obtain a recommended sleep case based on at least one of a sleep period, the number of midway awakening, and a sleep depth specified from the sleep information, and may obtain information indicating a representative posture in the recommended sleep case as safety posture information based on the posture information. The processing unit 311 may obtain the appearance frequency or the appearance period of each posture in the recommended sleep case or both of them from the posture information, and may determine, based on the obtained information, which posture is a representative posture representing the recommended sleep case. For example, the processing unit 311 acquires a sleep period and the number of midway awakening, and obtains a sleep in which the sleep period is equal to or longer than a predetermined time and the number of midway awakening is smaller than a predetermined threshold value as a recommended sleep case. However, the method for determining a recommended sleep case is not limited thereto. For example, in a case where sleep information indicating a sleep depth has been obtained, the processing unit 311 may obtain a recommended sleep case based on the length or ratio of a sleep period in which the sleep depth is equal to or deeper than a predetermined depth. It is known that the sleep depth can be determined from information such as brain waves, heartbeat, a respiratory rate, body movement, the number of roll-over, and vitals, and these methods are widely applicable to the embodiment. For example, the processing unit 311 obtains, based on the sleep information, a recommended sleep case indicating a sleep in which the length or ratio of a sleep period with a sleep depth equal to or deeper than a predetermined depth is equal to or larger than a predetermined second threshold value. In addition, any combination of a sleep period, the number of midway awakening, and a sleep depth is possible. Hereinafter, an example in which a recommended sleep case is obtained from a sleep period and the number of midway awakening will be described.

The processing unit 311 may obtain a non-recommended sleep case based on at least one of a sleep period, the number of midway awakening, and a sleep depth specified from sleep information, and may obtain, based on posture information, information indicating a representative posture of the non-recommended sleep case as risky posture information. As in the case of the recommended sleep case, the processing unit 311 obtains the appearance frequency or the appearance period of each posture in the non-recommended sleep case or both of them, and determines a representative posture based on the obtained information. The processing unit 311 acquires a sleep period and the number of midway awakening, and obtain a sleep in which the sleep period is shorter than a predetermined time, or the sleep period is equal to or longer than the predetermined period but the number of midway awakening is equal to or larger than a predetermined threshold value, as a non-recommended sleep case. However, even in the non-recommended sleep case, the processing unit 311 can use a sleep depth for the determination. For example, the processing unit 311 obtains, based on the sleep information, a non-recommended sleep case indicating a sleep in which the length or ratio of a sleep period with a sleep depth equal to or deeper than a predetermined depth is smaller than the predetermined second threshold value. In addition, any combination of a sleep period, the number of midway awakening, and a sleep depth is possible. Hereinafter, an example in which a non-recommended sleep case is obtained from a sleep period and the number of midway awakening will be described.

In this way, a representative posture in the case of a desirable sleep can be obtained as a safety posture. In addition, a representative posture in the case of an undesirable sleep can be obtained as a risky posture. That is, a safety posture and a risky posture can be obtained in consideration of effects of postures on a sleep.

FIG. 15 is a flowchart illustrating processing using sleep information. In step S301, the posture information acquisition unit 312 acquires posture information indicating a posture of the user. In step S302, the sleep information acquisition unit 314 acquires the sleep information of the user. For example, as described above, the seat sensor 810 outputs a determination result on a sleep/awake state of the user or sleep information indicating a sleep depth at predetermined intervals. The sleep information acquisition unit 314 acquires the sleep information from the seat sensor 810. The memory 320 stores the posture information and the sleep information.

In step S303, the processing unit 311 determines whether the sleep is ended. For example, the processing unit 311 determines, based on the sleep information, that the sleep is ended in a case where the state of the user is shifted from a sleep state to an awake state and a duration of the awake state becomes equal to or longer than a predetermined time. In other words, even in a case where the user is shifted to the awake state, when the awake state is ended before the predetermined time and shifted to the sleep state again, the processing unit 311 may determine that the sleep is not ended. Such awakening before the predetermined time is determined as midway awakening.

In a case where it is determined that the sleep is not ended (step S303: No), the processing returns to step S301, and acquisition of posture information and sleep information is continued.

In a case where it is determined that the sleep is ended (step S303: Yes), in step S304, the processing unit 311 obtains a sleep period and the number of midway awakening based on the sleep information. The sleep period is the length of a period from a timing at which the awake state is shifted to the sleep state as a starting point to a timing by which the awake state has continued for a predetermined time as an end point. Alternatively, the sleep period is a time obtained by subtracting the length of time of the awake state included midway from the length of the above described period. The number of midway awakening is the number of times of detection of the awake state during the above-described period. However, the determination of the end of the sleep (step S303) and the determination of the sleep period and the number of midway awakening (step S304) are not limited to the above, and can be modified in various ways. For example, the end of sleep may be determined in a case where the user performs an end-of-sleep operation using any device. The device here may be the bedside terminal device 200, or may be a mobile terminal device of the user, or may be another device.

In step S305, the processing unit 311 determines whether the above-described one sleep corresponds to a recommended sleep case that indicates a desirable sleep based on the sleep period and the number of midway awakening. Specifically, the processing unit 311 determines a sleep in which the sleep period is equal to or longer than a predetermined time and the number of midway awakening is smaller than a predetermined threshold value as a recommended sleep case.

In a case where it is determined that the target sleep corresponds to the recommended sleep case (step S305: Yes), in step S306, a representative posture is obtained from the posture information corresponding to the sleep, and the representative posture is determined as a safety posture. For example, the processing unit 311 may determine a posture that has most frequently appeared during a target period as a representative posture. For example, in a case where posture information can be acquired at regular intervals as in the example in FIG. 8, the processing unit 311 measures the number of times of detection of each posture from time-series posture information, and a posture with the largest number of times of detection (the highest appearance frequency) as a representative posture.

Alternatively, the processing unit 311 may obtain a representative posture based on an appearance period (in particular, a duration which is a consecutive appearance period). For example, the processing unit 311 obtains, for each of multiple postures, the maximum value of a time during which a posture has continued as the duration. Then, the processing unit 311 may determine a posture with the longest duration as a representative posture. In that case, even a posture with a high appearance frequency is not likely to be selected as a representative posture when the duration of the posture is short.

In a case where it is determined that the target sleep does not correspond to the recommended sleep case (step S305: No), in step S307, the processing unit 311 determines whether the above-described one sleep corresponds to a non-recommended sleep case that indicates an undesirable sleep based on the sleep period and the number of midway awakening. Specifically, the processing unit 311 determines a sleep for less than a predetermined time as a non-recommended sleep case. Alternatively, the processing unit 311 determines a sleep in which the number of midway awakening is equal to or larger than a predetermined threshold value as a non-recommended sleep case even when the sleep period is equal to or longer than the predetermined time.

In a case where it is determined that the target sleep corresponds to the non-recommended sleep case (step S307: Yes), in step S308, a representative posture is obtained from the posture information corresponding to the sleep, and the representative posture is determined as a risky posture. The process of obtaining a representative posture is the same as in step S306.

In a case where it is determined that the target sleep does not correspond to the non-recommended sleep case (step S307: No), the processing unit 311 ends the processing without specifying a safety posture and a risky posture. That is, the one sleep here may include an intermediate sleep case that is not a recommended sleep case or a non-recommended sleep case. However, one sleep may be classified as a recommended sleep case or a non-recommended sleep case without exception. In that case, in a case where it is determined that the target sleep does not correspond to the recommended sleep case (step S305: No), the target sleep corresponds to the non-recommended sleep case, and thus the processing in step S307 is omitted and the processing in step S308 is executed.

### 2.4.2 Feedback (Nasal Blockage)

The health information acquisition unit (the feedback information acquisition unit 315) of the information processing system 10 according to the embodiment may acquire feedback information indicating a determination result of the physical condition by the user at get-up time as health information. Then, the processing unit 311 obtains representative posture information indicating a representative posture during one sleep based on the posture information. In a case where it is determined based on the feedback information that the user felt a change in the physical condition at get-up time from the one sleep, the processing unit 311 determines the representative posture information as risky posture information or safety posture information. Similar to the recommended sleep case or the like, the processing unit 311 obtains, based on at least one of the appearance frequency and the appearance period of each posture during the one sleep, a representative posture during the one sleep. Note that the processing unit 311 may obtain a representative posture based on the posture information of the entirety of the one sleep, as in the examples of the recommended sleep case and the non-recommended sleep case. However, considering that the physical condition at get-up time is an analysis target, the processing unit 311 may obtain a representative posture based on the posture information for a time close to the get-up time in the one sleep. For example, the processing unit 311 obtains a representative posture based on the posture information for one hour before getting up.

Accordingly, it is possible to specify a risky posture or a safety posture based on a subjective feeling of the user. That is, in a case where the user has had a sleep with feeling of comfort at get-up time, a posture that can be a cause for the comfort can be specified as a safety posture, or in a case where the user has had a sleep with feeling of discomfort at get-up time, a posture that can be a cause for the discomfort can be specified as a risky posture. Note that the feedback information is not limited to that input by the user. For example, a person around the user such as a family member of the user, a care giver, and a medical staff member may hear the physical condition from the user, and then may input the hearing result as the feedback information in place of the user. Alternatively, these persons around the user may estimate the physical condition of the user based on the facial color, the responding voice, or the like of the user, and then may input the estimation result as the feedback information in place of the user.

The feedback information here may be information about nasal blockage. That is, after getting up, the user inputs presence or absence of nasal blockage (or a degree of worsening or improvement) during a sleep using the terminal device 600 or the bedside terminal device 200, and the server system 300 acquires the input result as the feedback information.

In a case where it is determined that the nasal blockage of the user has been worsened based on the feedback information, the processing unit 311 performs a process of determining the representative posture information as risky posture information that has an adverse effect on the nasal blockage. In contrast, in a case where it is determined that the nasal blockage of the user has been improved based on the feedback information, the processing unit 311 performs a process of determining the representative posture information as safety posture information that has a favorable effect on the nasal blockage. The processing unit 311 may perform the both processes. Accordingly, it is possible to suggest to the user that postures may affect the nasal blockage. The user is likely to recognize the nasal blockage at get-up time, but is less likely to recognize the nasal blockage during a sleep. In this respect, in the method of the embodiment, since a relationship between a representative posture during a sleep and nasal blockage can be determined, it is possible to enable the user to recognize the possibility that the nasal blockage during the sleep can be improved by posture changing.

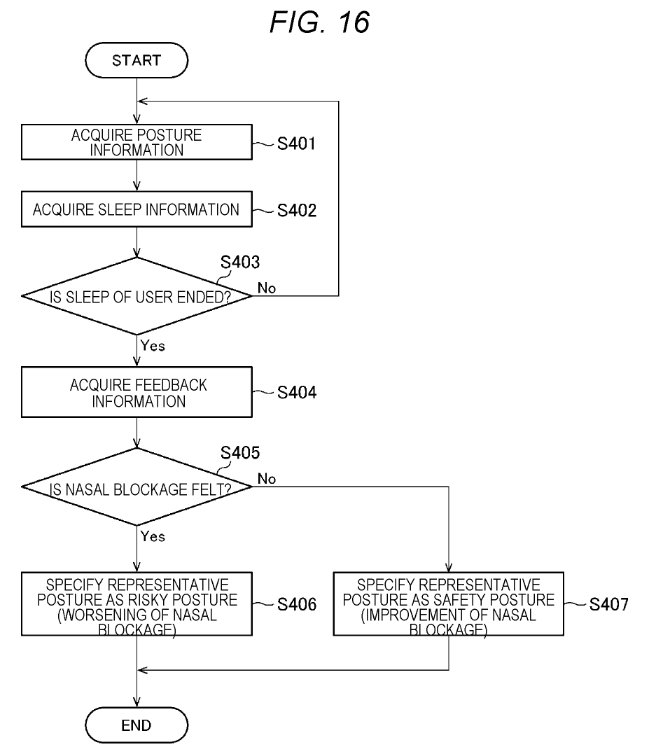

FIG. 16 is a flowchart illustrating processing in a case where the feedback information is nasal blockage. In step S401, the posture information acquisition unit 312 acquires posture information indicating a posture of the user. In step S402, the sleep information acquisition unit 314 acquires the sleep information of the user. In step S403, the processing unit 311 determines whether the sleep is ended. In a case where it is determined that the sleep is not ended (step S403: No), the processing returns to step S401, and acquisition of the posture information and the sleep information is continued. The processing in steps S401 to S403 is the same as the processing in steps S301 to S303 in FIG. 15.

In a case where it is determined that the sleep is ended (step S403: Yes), in step S404, the feedback information acquisition unit 315 acquires feedback information. For example, the feedback information acquisition unit 315 may perform a process of causing the display of the terminal device 600 or the like to display a screen for prompting an input related to nasal blockage. The feedback information acquisition unit 315 acquires the result of user's input on the terminal device 600 or the like as feedback information.

In step S405, the processing unit 311 determines whether the user felt the nasal blockage based on the feedback information. In a case where it is determined that the user felt the nasal blockage (step S405: Yes), in step S406, a representative posture is obtained from the posture information corresponding to the sleep, and the representative posture is determined as a risky posture that worsens the nasal blockage. The process of obtaining a representative posture is the same as in the example described above using FIG. 15, and thus the detailed description thereof will be omitted.

In a case where it is determined that the user did not feel the nasal blockage (step S405: No), in step S407, the processing unit 311 obtains a representative posture from the posture information corresponding to the sleep, and determines the representative posture as a safety posture that improves the nasal blockage.

### 2.4.3 Defecation

The health information acquisition unit (defecation information acquisition unit 316) of the information processing system 10 according to the embodiment may acquire defecation information related to the defecation of the user as health information. The processing unit 311 obtains representative posture information indicating a representative posture which is a representative posture during one sleep based on the posture information. In a case where it is determined based on the defecation information that defecation is present during the one sleep or on the next day, the processing unit 311 determines the representative posture information as defecation recommendation posture information which indicates a posture suitable for defecation. The processing unit 311 obtains, based on at least one of the appearance frequency and the appearance period of each posture during the one sleep, a representative posture during the one sleep.

For example, in the case of a serious patient who is continuously in a bedridden state, it is not possible to maintain constant defecation due to the influence of a sleeping posture or medication, and constipation may become a problem. In this case, the state of defecation may be improved by selecting a suitable sleeping posture. In this respect, in the method of the embodiment, a posture suitable for constipation improvement can be automatically specified based on defecation information and a representative posture.

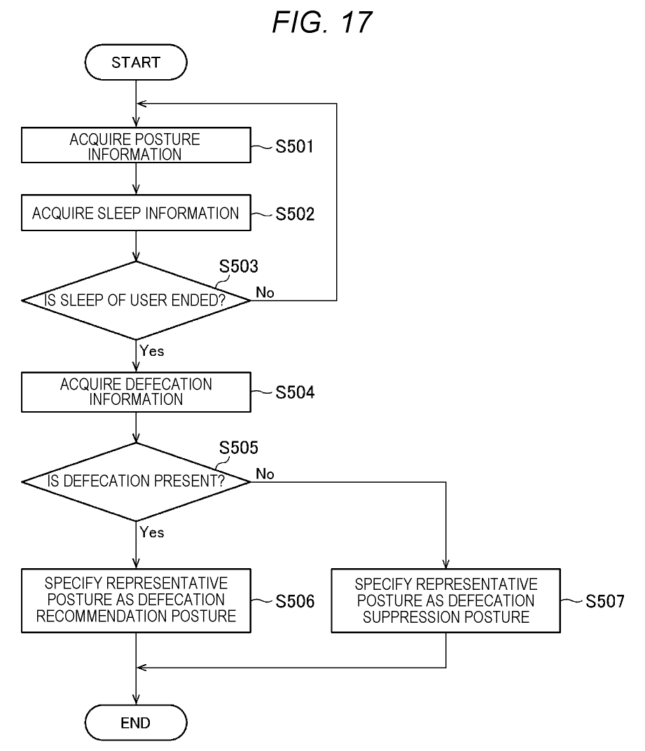

FIG. 17 is a flowchart illustrating processing using defecation information. In step S501, the posture information acquisition unit 312 acquires posture information indicating a posture of the user. In step S502, the sleep information acquisition unit 314 acquires the sleep information of the user. In step S503, the processing unit 311 determines whether the sleep is ended. In a case where it is determined that the sleep is not ended (step S503: No), the processing returns to step S501, and acquisition of the posture information and the sleep information is continued. The processing in steps S501 to S503 is the same as the processing in steps S301 to S303 in FIG. 15.

In a case where it is determined that the sleep is ended (step S503: Yes), in step S504, the defecation information acquisition unit 316 acquires defecation information. For example, the defecation information acquisition unit 316 may acquire defecation information indicating presence or absence of defecation by referring to a care record and a nursing record stored in the electronic medical record server 400. In addition, since the defecation on the next day is not necessarily present in the morning, the acquisition of the defecation information may be executed at a timing such as the next night or the day after next.

In step S505, the processing unit 311 determines the presence or absence of the defecation of the user based on the defecation information. In a case where it is determined that the defecation is present (step S505: Yes), in step S506, a representative posture is obtained from the posture information corresponding to the sleep, and the representative posture is determined as a defecation recommendation posture that improves the defecation. The process of obtaining a representative posture is the same as in the example described above using FIG. 15, and thus the detailed description thereof will be omitted.

In a case where it is determined that the defecation is absent (step S505: No), in step S507, the processing unit 311 obtains a representative posture from the posture information corresponding to the sleep, and determines the representative posture as a defecation suppression posture that suppresses the defecation. Note that the processing in step S507 may be omitted, and only a process of obtaining a defecation recommendation posture may be executed.

In the foregoing, the examples in which the defecation recommendation posture and the defecation suppression posture are obtained based on the posture information and the defecation information corresponding to one sleep have been described, but these examples are not intended to be limitations. For example, the memory 320 may accumulate information in which a representative posture and defecation information are associated with each other for each night for a predetermined period (e.g., 30 days). The processing unit 311 specifies a posture having a positive trend for defecation as a defecation recommendation posture. Similarly, the processing may be performed using an accumulation result for a predetermined period in other examples such as the above described sleep case and feedback (nasal blockage in a narrow sense).

Note that the process of acquiring the defecation information may be changed depending on whether independent excretion is possible. For example, in a case where the user can independently move to a toilet for excretion, the defecation information may be included in the above-described feedback information. That is, the user inputs the presence or absence of defecation, whereby the defecation information is obtained. On the other hand, in the case of the user who has difficulty in independent excretion, the defecation information may be information input by a care worker or a nurse, or may be information detected by a sensor (such as an odor sensor) disposed at a diaper or the bed 100. Alternatively, the weight of a diaper may be measured before disposal, and the defecation information may be detected based on a change in the weight from the unused state.

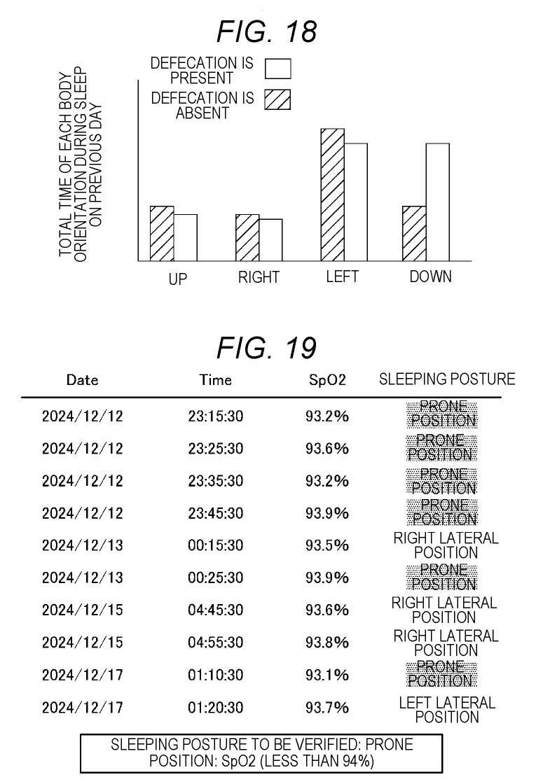

The processing unit 311 may obtain a total time indicating a total of time during which each posture has appeared. FIG. 18 is a graph in which the total time of each posture during a sleep on the previous day and presence or absence of defecation are associated with each other. The horizontal axis of the graph in FIG. 18 represents postures and the vertical axis represents a total time of each posture in accordance with presence or absence of defecation. Note that up, right, left, and down represent a supine position, a right lateral position, a left lateral position, and a prone position, respectively.

As illustrated in FIG. 18, in the case of the supine position, total times of both a sleep during which defecation was present and a sleep during which defecation was absent are short, and a difference in total time between the both sleeps is also small. The same applies to the right lateral position. The left lateral position appeared for a long time during the sleep, but a difference in time between a case where defecation was present and a case where defecation was absent is small, and the time during which the left lateral position appeared tends to be longer in the case where defecation was absent. Therefore, the processing unit 311 determines that the supine position, the right lateral position, and the left lateral position contribute little to defecation.

In contrast, in the case of the prone position, the total time of a sleep during which defecation was present is recognizably longer than that of a sleep during which defecation was absent. Therefore, it can be determined that defecation may be improved by taking the prone position for a long time. That is, the processing unit 311 may determine the prone position as a defecation recommendation posture.

For example, in a case where a posture in which the total time with defecation is longer than the total time without defecation by a predetermined time or more (or by a predetermined ratio or more) and the total time with defecation is equal to or longer than a predetermined threshold value is detected, the processing unit 311 determines the posture as a representative posture that improves defecation, that is, a defecation recommendation posture. In this way, it is possible to determine with high accuracy whether a target posture contributes to defecation by comparison with the total time without defecation.

Although a description has been made on defecation (excretion) above, the method of the embodiment can be extended to urination. In this case, the processing unit 311 may obtain a urination recommendation posture that promotes urination or a urination suppression posture that suppresses urination, or the both postures based on information on presence or absence of urination, the number of times of urination, or the like.

### 3. Utilization of Posture

Hereinafter, processing using a risky posture, a safety posture, and the like will be described.

### 3.1 Display Processing

Display of Determination Results of Risky Posture, Safety Posture, and Specific Risky Posture

The processing unit 311 (analysis display processing unit) may perform display processing of a risky posture or display processing of a safety posture, or the both display processing. In addition, the processing unit 311 may display information about presence or absence of a risk or a degree of a risk that is a determination result on a specific risky posture. Hereinafter, a determination result on a specific risky posture will be denoted as specific risk determination information.

For example, the processing unit 311 causes the memory 320 to store risky posture information, safety posture information, and specific risk determination information. Alternatively, the processing unit 311 may cause the memory of the electronic medical record server 400 to store these pieces of information. Recording to the electronic medical record server 400 means registering to the electronic medical record.

The processing unit 311 may perform a process of causing the display of each device included in the information processing system 10 in FIG. 1 to display risky posture information, safety posture information, and/or specific risk determination information. The display here may be the display of the electronic medical record server 400, may be the display of the station terminal device 500, or may be the display of the terminal device 600. This enables a medical doctor, a nurse, or the like to refer to the risky posture information, the safety posture information, and/or the specific risk determination information. Since these pieces of information are reflected into settings of the bed 100 and the mattress 170 and daily life guidance, avoidance of a risky posture or recommendation of a safety posture during a sleep become possible. For example, in order to avoid a risky posture, a staff member can take measures such as setting of the bed 100, position adjustment of air mattress, and selection of a pillow or a support cushion for postural change. Further, in a case where it is determined that a risk of a specific risky posture is high, it is possible to avoid the specific risky posture during a sleep.

Note that specific measures may be recommended from the processing unit 311 to a staff member without being entrusted to the staff member. For example, the memory 320 may store data in which a risky posture and measures for avoiding the risky posture are associated with each other. In a case where a risky posture is specified, the processing unit 311 performs a process of displaying measures against the risky posture. The measures here include setting of a bed, position adjustment of an air mattress, selection of a pillow or a support cushion for postural change, execution of an inspection, and the like.

The risky posture information, the safety posture information, and/or the specific risk determination information may be transferred to the bedside terminal device 200 to be displayed for a user (patient). Further, in a case where communication with a patient terminal not illustrated in FIG. 1 is possible, the processing unit 311 may perform a process of causing the display of the patient terminal to display the risky posture information and the like. The patient terminal may be a smartphone for personal use by a patient, a wristwatch-type device for acquiring vital information, or the like.

In the bedside terminal device 200 or the like, the risky posture information may be displayed together with a medical staff inquiry button. The inquiry button is a button for inquiring of a medical staff member about explanation of a situation in which a posture is specified as a risky posture and guidance on bed settings (including setting of a bed, position adjustment of an air mattress, selection of a pillow and a support cushion for postural change) for avoiding the posture. For example, in a case where the inquiry button is pressed, the bedside terminal device 200 transmits information for specifying a target user, the risky posture information associated with the inquiry button, and information indicating that an inquiry about explanation and settings is required to the terminal device 600 of a staff member.

### Display of Defecation Recommendation Posture and the like

In a case where a defecation recommendation posture is obtained (FIGS. 17 to 18), display in accordance with the defecation state of the user may be performed. For example, in a case where the target user suffers from constipation, displaying a defecation recommendation posture that improves defecation enables a staff member to set bedding so that a posture with easy defecation can be easily taken. Alternatively, when determining that the target user suffers from constipation by referring to the electronic medical record server 400, the processing unit 311 may perform a process of recommending specific bedding.

However, a display target is not limited to a defecation recommendation posture. For example, in a period such as night-time in which an interval between diaper changes is relatively long, it may be preferable to suppress defecation in a diaper. Therefore, in a case where the user is a care target person who wears a diaper, the processing unit 311 may perform display recommending a defecation suppression posture in a time period during which defecation in the diaper is not desirable. This display may be performed in the above-described time period, or may be performed before the above-described time period (e.g., a time period of preparation for sleeping). Also in this case, the processing unit 311 may perform a process of recommending specific bedding as in the example of a defecation recommendation posture.

Similarly, in a case where the user suffers from a urination disorder, the processing unit 311 performs a process of displaying a urination recommendation posture. In contrast, in a case where the user suffers from enuresis or midway awakening to use a toilet, the processing unit 311 performs a process of displaying a urination suppression posture. This makes it possible to appropriately prompt promotion or suppression of defecation and urination in accordance with the state of the user.

The processing unit 311 may perform a process of estimating an occurrence timing or an occurrence probability of a next defecation event based on a posture indicated by posture information and the already acquired defecation recommendation posture, defecation suppression posture, urination recommendation posture, and urination suppression posture. Then, the processing unit 311 performs a process of suggesting execution of dietary modification and massage for defecation improvement based on the estimation result. In a case where the target user is a care target person who has a difficulty in independently moving to a toilet, the processing unit 311 may execute, based on an estimation result, processing such as adjustment of the number of times and timing of using a toilet, determination on wearing or removing a diaper, and selection of diaper volume, and may present an execution result to a staff member.

### Highlight Display of Risky Posture in List

In a case where a risky posture is specified, the risky posture may be displayed in association with other information. For example, as described above using FIG. 8 and the like, the memory 320 may store time information indicating acquisition timings of posture information and health information (e.g., vital information) in association with the posture information and the health information. In a case where risky posture information is acquired, the processing unit 311 may determine the state of the user based on the health information acquired in a predetermined display target period, and may perform control of causing a display to display a screen including data (e.g., list data to be described below using FIG. 19) in which posture information and time information are associated with each other for the health information that is determined as a bad state. At this time, the processing unit 311 causes the display to display the posture information on the screen in a mode in which an item corresponding to the risky posture information can be distinguished from items indicating other postures. This makes it possible to present the risky posture information in a visually-recognizable mode in data in which worsening of the health condition is suspected. As a result, a staff member can verify the degree of influence of a posture (a risky posture in a narrow sense) on the state of the user.

FIG. 19 is a diagram illustrating an example of a display screen including list data in which posture information and time information are associated with each other for vital information in which the state is determined to be bad. FIG. 19 illustrates an example in which SpO2 is acquired as the vital information, and the state of the user is determined as bad in a case where the value of SpO2 is less than 94%. As described above using FIG. 8 and the like, the time information and the posture information are associated with the vital information. Therefore, the processing unit 311 extracts the vital information in which SpO2 is less than 94% and displays the time information and the posture information associated with the vital information. FIG. 19 illustrates an example in which the time information is information of year, month, day, hour, and minute, and the posture information is information indicating any of a supine position, a right lateral position, a left lateral position, and a prone position, but various modifications can be made as described above.

Here, it is assumed that the processing unit 311 performs the processing described above using FIG. 6, thereby acquiring a determination result indicating that the prone position is a risky posture. In this case, as illustrated in FIG. 19, the processing unit 311 displays the posture information such that items corresponding to the prone position can be distinguished from items corresponding to other postures. FIG. 19 illustrates an example of highlight display in which a background color is changed. However, it is sufficient that the risky posture is recognizable, and various modifications such as a change in character color or character size, a change in font, and icon display in the vicinity can be made to a specific display mode. By displaying such a screen, it is possible to prompt a staff member to verify the degree of influence of a posture (a risky posture in a narrow sense) on worsening of the state of the user. In addition, it is also possible to prompt the staff member to execute a necessary inspection at an early stage.

FIG. 19 illustrates an example in which a risky posture is displayed in a highlight manner on the assumption that the risky posture is already specified, but this is not a limitation. For example, as in FIG. 19, the processing unit 311 may perform a process of extracting data in which the state is determined to have been worsened based on the vital information, and in a case where a specific posture exceeds a predetermined ratio (e.g., more than half) in the extracted data, may perform a process of displaying the posture in a highlight manner.

Note that FIG. 19 illustrates an example in which extraction processing of a part of data based on the vital information and highlight display of a risky posture are performed, but this is not a limitation. For example, the processing unit 311 may display time-series data in which time information, vital information, and posture information are associated one another as-is. For example, the processing unit 311 performs a process of causing the displays of the bedside terminal device 200, the electronic medical record server 400, the station terminal device 500, the terminal device 600 and the like to display the list data illustrated in FIG. 8, FIGS. 11 and 13.

### Display of Analysis Result

The processing unit 311 may perform a process of causing a display to display a result of the analysis processing illustrated in step S104 in FIG. 6 or step S204 in FIG. 10. The result of the analysis processing may be information in which multiple postures can be compared with each other as illustrated in FIGS. 9A to 9D. Alternatively, the result of the analysis processing may be information indicating a feature point in the time-series change of vital information as illustrated in FIG. 12. The feature point may be a point indicating improvement of the state or may be a point indicating worsening of the state, and is determined by comparison between a value of the vital information and a predetermined threshold value. Alternatively, the result of the analysis processing may be information indicating changes in statistic values of the vital information obtained for each predetermined period as illustrated in FIGS. 14A to 14C. Using the result of the analysis processing enables essential and easy-to-understand display as compared with using list data in FIG. 8 or the like as-is, for example.

However, in order to enable a staff member or the like who views a content of an analysis result to easily understand the content, it is important to present a correspondence relationship indicating when, in which posture, and what value the vital information showed. In a case where a graph (in particular, a two-dimensional graph) that can be visually and easily understood is used, it is not easy to clearly indicate a correspondence relationship between three values.

Thus, the processing unit 311 may perform a process of causing a display to display a screen including a planar graph in which an axis corresponding to a value of any one of health information (e.g., vital information), posture information, and time information is set as a first axis, and an axis corresponding to a value of one of other pieces of information is set as a second axis intersecting the first axis, and superimpose and display a value of the remaining one of the health information, the posture information, and the time information on the planar graph. This enables display that realizes both easiness of understanding and suppression of missing information quantity.

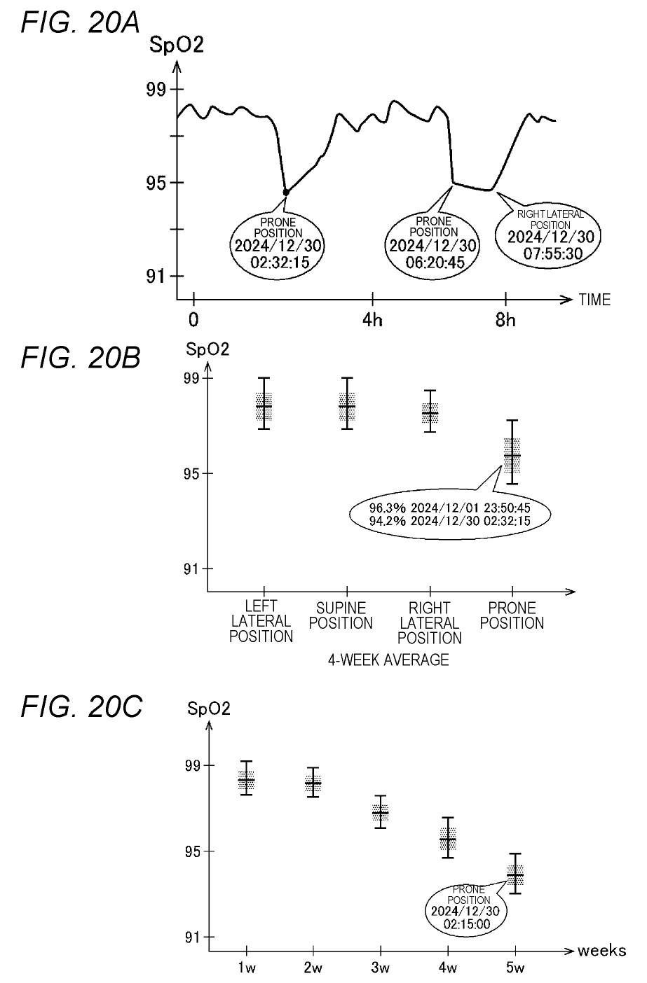

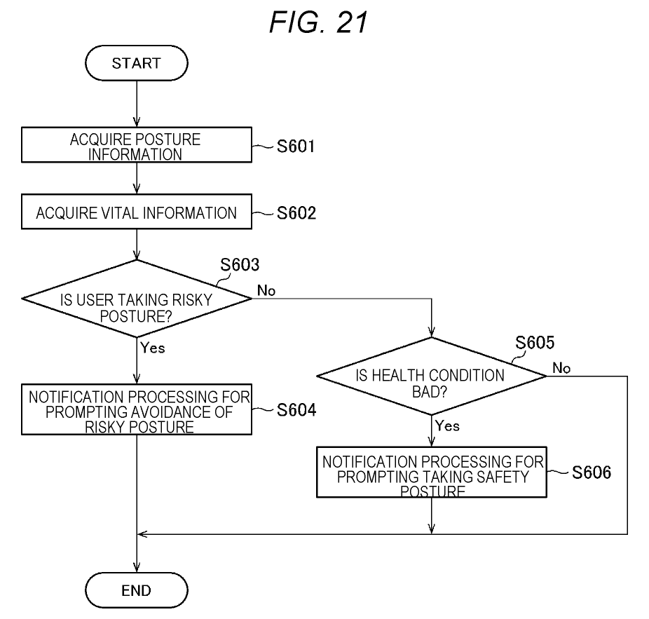

FIGS. 20A to 20C are diagrams each illustrating an example of a screen displaying an analysis result. FIGS. 20A to 20C illustrate examples in which the vital information is SpO2, but other vital information may be used. In addition, a staff member or the like may be able to select which vital information is to be displayed. For example, when receiving an operation of selecting specific vital information, the processing unit 311 causes a display to display a result of analysis processing including the selected vital information.

As illustrated in FIG. 20A, the processing unit 311 may display, as a result of the analysis processing, a screen including a time-series graph in which time information is indicated on the horizontal axis and vital information is indicated on the vertical axis. Then, in the case of a significant change in the value of the vital information, the processing unit 311 superimposes and displays posture information corresponding to a relevant time stamp in a balloon form. Accordingly, the vital information, the time information, and the posture information at the feature point can be displayed in an easy-to-understand mode. Note that the balloon display of posture information may be configured to be superimposed and displayed on any time of day selected by the user with a cursor.

As illustrated in FIG. 20B, the processing unit 311 may display, as a result of the analysis processing, a screen including a graph (box chart) in which posture information is indicated on the horizontal axis and statistic values of vital information are indicated on the vertical axis. Then, in a case where a statistic value of the vital information satisfies a predetermined condition, the processing unit 311 superimposes and displays time information corresponding to the statistic value with a balloon. Here, in consideration of the fact that the vital information indicates statistic values, the vital information at predetermined one timing indicating a significant value change may be displayed in the balloon. Accordingly, the vital information, the time information, and the posture information can be displayed in an easy-to-understand mode.

As illustrated in FIG. 20C, the processing unit 311 may display, as a result of the analysis processing, a screen including a graph (box chart) in which time information is indicated on the horizontal axis and statistic values of vital information are indicated on the vertical axis. The horizontal axis here may be on a weekly basis. Then, in a case where a statistic value of the vital information satisfies a predetermined condition, the processing unit 311 superimposes and displays posture information corresponding to the statistic value with a balloon. Here, in consideration of the fact that the horizontal axis is on a weekly basis, a time stamp indicating predetermined one timing indicating a significant value change of SpO2 may be displayed in the balloon. Accordingly, the vital information, the time information, and the posture information can be displayed in an easy-to-understand mode.

FIGS. 20A to 20C illustrate examples of screens displaying the results of the analysis processing, and various modifications can be made to a specific display mode. For example, display of detailed information is not limited to superimposing and displaying with a balloon. In addition, a display screen may employ various display modes such as table data, a bar graph, a polygonal line graph, a histogram, a box and whisker plot, and a violin plot.

### 3.2 Notification Processing

In a case where risky posture information is acquired, the processing unit 311 may perform notification processing based on the risky posture information in a subsequent period. The notification processing here may be a push notification.

For example, in a case where risky posture information is obtained based on posture information and health information in a first period, the processing unit 311 performs notification processing in a second period after the first period when determining that the user is taking a posture corresponding to the risky posture information based on the posture information of the user. A notification destination here may be the terminal device 600 of a staff member in charge of the target user. The terminal device 600 and the like outputs a notification result using a mode such as screen display, vibration, sound output and the like. Accordingly, it is possible to prompt the staff member to change the posture of the user. The notification destination may be a device to which the user (patient) can refer, such as the bedside terminal device 200 and the like. For example, in a case where a risky posture is kept for a predetermined time or more, the processing unit 311 may cause the display of the bedside terminal device 200 to perform highlight display and may cause a wearable type measuring device worn by the patient to make a notification by vibration or voice. This enables the patient to enhance his/her consciousness and proactively avoid the risky posture.

In a case where safety posture information is obtained based on posture information and health information in the first period and it is determined that the state of the user has been worsened based on the vital information of the user in the second period after the first period, the processing unit 311 may perform notification processing for prompting a change to a safety posture. With this configuration, in a case where worsening of the state is detected, it is possible to prompt a postural change for suppressing the worsening. The notification destination may be a device used by a staff member or a device used by the user.

FIG. 21 is a flowchart illustrating notification processing. It is assumed that, before this processing, the processing illustrated in FIG. 6 is executed, and risky posture information is acquired in step S106. Similarly, it is assumed that, before the processing illustrated in FIG. 21, the processing illustrated in FIG. 10 is executed, and safety posture information is acquired in step S206.

In step S601, the posture information acquisition unit 312 acquires posture information. In step S602, the vital information acquisition unit 313 acquires vital information. That is, in the method of the embodiment, posture information and vital information may be acquired on an ongoing basis even after acquisition of risky posture information and the like.

In step S603, the processing unit 311 compares the real-time posture information acquired in step S601 and preliminarily obtained risky posture information, thereby determining whether the user is taking a risky posture.

In a case where it is determined that the user is taking a risky posture (step S603: Yes), in step S604, the processing unit 311 performs notification processing for avoiding the risky posture. Specifically, as described above, the processing unit 311 performs a process of transmitting notification information via the communicator 330 to other devices such as the terminal device 600, the bedside terminal device 200, and the like. The notification information is information for prompting a change to a posture other than the current posture (the risky posture).

Note that the determination processing in step S603 is not limited to determination of only the posture at predetermined one timing. For example, when determining that a risky posture is kept for a predetermined time or more (e.g., 30 minutes or more), the processing unit 311 may determine that the user is taking the risky posture.

In a case where it is determined that the user is not taking a risky posture (step S603: No), in step S605, the processing unit 311 determines whether the state of the user has been worsened based on the vital information. The processing is, for example, comparison processing between a value of the real-time vital information acquired in step S602 and a given threshold value.

In a case where it is determined that the state of the user has been worsened (step S605: Yes), in step S606, the processing unit 311 performs notification processing for prompting a change to a safety posture. Specifically, as described above, the processing unit 311 performs a process of transmitting notification information via the communicator 330 to other devices such as the terminal device 600, the bedside terminal device 200, and the like. The notification information is information for prompting a change to a known safety posture.

### 3.3 Control of Bed and The Like

In the foregoing, descriptions have been made on examples of prompting postural change or setting of the bed 100 and the like by performing display processing or notification processing. However, the processing unit 311 of the embodiment may control the bed 100 or the mattress 170 (air mattress) to automatically set a position to enable an appropriate posture.

For example, the bed 100 according to the embodiment may have a postural change function of changing the posture of a patient (for a purpose of pressure ulcer or contracture prevention, including back raising, knee raising, foot raising, or postural change in a lateral direction, for example) by adjusting angles of sections. The processing unit 311 determines a suitable position of the bed 100 by adjusting section angles based on specified risky posture information or safety posture information.

Alternatively, an air mattress used in combination with the bed 100 may be equipped with support air cells for postural change, and the above-described postural change function may be implemented by inflation and deflation of the support air cells. The processing unit 311 regulates the postural change function of the air mattress by controlling inflation and deflation of each of the air cells based on specified risky posture information or safety posture information.

For example, in a case where risky posture information is obtained based on posture information and health information in a first period, and when determining that the user is taking a posture corresponding to the risky posture information based on the posture information of the user in a second period after the first period, the processing unit 311 may perform control of moving a movable part of at least one of the bed 100 and the mattress 170 used by the user so as to change the posture of the user. Accordingly, it is possible to cause the user to avoid taking a risky posture without an action by a staff member or the user.

Alternatively, in a case where safety posture information is obtained based on posture information and health information in the first period, and when determining that the state of the user has been worsened based on the health information of the user in the second period after the first period, the processing unit 311 may perform control of moving a movable part of at least one of the bed 100 and the mattress 170 used by the user so as to change the posture of the user to a posture corresponding to the safety posture information. Accordingly, it is possible to cause the user to take a safety posture without an action by a staff member or the user.

Similarly, the control of the bed 100 and the like is possible in place of display processing also in a case where a defecation recommendation posture or the like is obtained. For example, in a case where the user suffers from constipation, the processing unit 311 may automatically control the bed 100 or the like such that a defecation recommendation posture can be easily taken. Further, in a case where the user suffers from enuresis or midway awakening to use a toilet, the processing unit 311 may automatically control the bed 100 or the like such that a urination suppression posture can be easily taken. Furthermore, in a case where the user wears a diaper, the processing unit 311 may automatically control the bed 100 or the like such that a defecation suppression posture can be easily taken in a time period during which defecation in the diaper is not desirable.

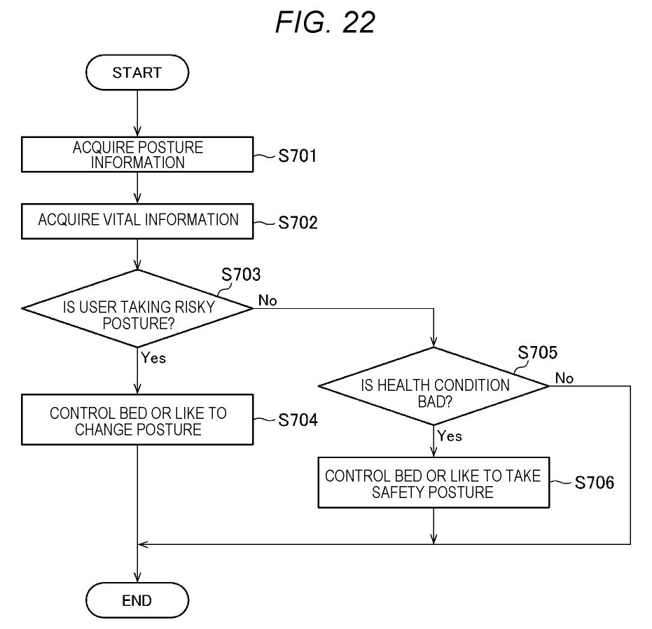

FIG. 22 is a flowchart illustrating automatic control of the bed 100 or the like. It is assumed that, before this processing, the processing illustrated in FIG. 6 is executed, and risky posture information is acquired in step S106. Similarly, it is assumed that, before the processing illustrated in FIG. 21, the processing illustrated in FIG. 10 is executed, and safety posture information is acquired in step S206.

In step S701, the posture information acquisition unit 312 acquires posture information. In step S702, the vital information acquisition unit 313 acquires vital information.

In step S703, the processing unit 311 compares the real-time posture information acquired in step S701 and preliminarily obtained risky posture information, thereby determining whether the user is taking a risky posture.

In a case where it is determined that the user is taking a risky posture (step S703: Yes), in step S704, the processing unit 311 controls the bed 100 or the like so as to avoid the risky posture. For example, the memory 320 may store information in which an angle of each section of the bed 100 and a posture that the user can easily take in a case where each section is set to the angle are associated with each other. The processing unit 311 specifies section angles corresponding to a posture other than the risky posture based on the above information, and transmits control information for obtaining the section angles to the bed 100. The same applies to a case where an air mattress is used, and the memory 320 may store information in which a degree of inflation of each air cell of the mattress 170 and a posture that the user can easily take in a case where each air cell is set to the degree of inflation are associated with each other. The processing unit 311 specifies the degree of inflation of each air cell corresponding to a posture other than the risky posture based on the above information, and transmits control information for obtaining the degree of inflation to the mattress 170. In addition, the processing unit 311 may execute the processing based on a combination of the section angles of the bed 100 and the degrees of inflation of the air cells of the mattress 170.

Note that, in a case where there are multiple postures other than a risky posture, the processing unit 311 may select, as a change destination posture, a safety posture, a posture with which the degree of change from the current posture is small, or a posture whose appearance frequency is high based on the history of past posture information.

In addition, the determination processing in step S703 is not limited to determination of only a posture at a predetermined one timing, but may determine a duration as in the processing in step S603 in FIG. 21.

In a case where it is determined that the user is not taking a risky posture (step S703: No), in step S705, the processing unit 311 determines whether the state of the user has been worsened based on the vital information.

In a case where it is determined that the state of the user has been worsened (step S705: Yes), in step S706, the processing unit 311 control the bed 100 or the like such that the user takes a safety posture. For example, the processing unit 311 specifies section angles or degrees of inflation of air cells associated with a safety posture or a combination of the both, and transmits the specified information to the bed 100 or the like via the communicator 330.

The display processing, the notification processing, and the control processing of the bed 100 or the like described above are not limited to be executed individually, but two or more of them may be executed in combination.

### 4. Modification Example

In the foregoing, an example in which the posture of the user acquired using the imaging device 700 or a motion sensor is used as posture information has been described. In other words, the above-described posture information is information indicating a result of directly detecting the posture of the user. However, the posture information of the embodiment is not limited thereto.

For example, the information processing system of the embodiment may include a movement information acquisition unit 317 that acquires movement information indicating the position and posture of a movable part of the bed 100 or the mattress 170 used by the user (FIG. 4). In a case where the information processing system corresponds to the server system 300, the movement information acquisition unit of the information processing system corresponds to the movement information acquisition unit 317 in FIG. 4.

The movement information here may be, for example, information indicating the state of each section of the bed 100, and may be information indicating a back raising amount, a knee raising amount, a foot raising amount, and a lateral inclination amount. The back raising amount may be a specific angle, or information indicating stages such as NO (0°), LOW (up to 15°), MIDDLE (15 to 45°), and HIGH (45 to 80°). Similarly, the lateral inclination amount may be a specific angle, or information indicating stages such as LEFT HIGH (-15° or more), LEFT LOW (-15 to 0°), NO, RIGHT LOW (0 to 15°), and RIGHT HIGH (15° or more). Alternatively, the movement information may be information indicating the degree of inflation of each air cell of the mattress 170.

The movement information acquisition unit 317 acquires movement information from the bed 100 and the mattress 170 at regular intervals. For example, movement information may be acquired in association with time information (time stamp), as in the case of posture information and vital information. The memory 320 stores posture information, vital information, and movement information in association with each other. Association is made using time stamps, for example.

The processing unit 311 may determine the state of the user based on health information, and may perform at least one of (1) a process of obtaining risky posture information by a combination of posture information and movement information associated with the health information in which the state of the user is determined as a first state with a worsening risk and (2) a process of obtaining safety posture information by a combination of posture information and movement information associated with the health information in which the state of the user is determined as a second state with a relatively low worsening risk.

Accordingly, it is possible to extend the posture information in the embodiment to combinations of results of direct detection of the posture of the user (posture information in a narrow sense) and control information (movement information) of peripheral devices that affect the posture of the user. That is, risky posture information and safety posture information may be information indicated by a combination of posture information in a narrow sense and movement information. Also in that case, the processing unit 311 can execute the above-described display processing, notification processing and automatic control processing of the bed 100 or the like.

In the present modification example, risky posture information includes movement information. Therefore, in a case where the movable part 160 of the bed 100 reaches a state corresponding to risky posture information as a result of a position operation of the bed 100, the processing unit 311 may perform a warning display (warning lamp display, GUI warning display, or warning sound notification) on the display of the bed 100 and the bedside terminal device 200, or may make a notification to the station terminal device 500 and the terminal device 600 to cause them to execute warning processing.

The bed 100 that has been notified of a extended risky posture state may, in a case where a position operation of the bed is input and the position operation leads to a risky position state, prohibit the position operation so as to avoid a risky bed position. In that case, it is also possible to clearly indicate that the bed position operation has been automatically prohibited, on the display of the bed 100 and the bedside terminal device 200.

Note that, although the embodiment has been described in detail above, it will be readily understood by those skilled in the art that various modifications can be made without departing from the novel matters and effects of the embodiment. Accordingly, all of these modifications shall fall within the scope of the present disclosure. For example, the term that is mentioned at least once in the specification or drawings with a different term that is a broader term or synonym may be replaced by that different term at any point in the specification or drawings. In addition, all combinations of the embodiment and the modifications shall fall within the scope of the disclosure. Further, the configuration and operation of the information processing system, the server system, the terminal device, the bedside terminal device, and the like are not limited to those described in the embodiment, and various modifications can be made.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the invention.

What is claimed is:

1. An information processing system comprising:
a posture information acquisition unit configured to acquire posture information indicating a posture of a user;
a health information acquisition unit configured to acquire health information indicating a health condition of the user;
a memory configured to store the posture information and the health information in association with each other; and
a processing unit configured to determine a state of the user based on the health information, and perform at least one of
(1) a process of obtaining risky posture information based on the posture information associated with the health information in which the state of the user is determined as a first state with a worsening risk being present, and
(2) a process of obtaining safety posture information based on the posture information associated with the health information in which the state of the user is determined as a second state with the worsening risk being relatively low.

2. The information processing system according to claim 1, wherein
in a case where the user is subjected to postural drainage which is care for removing phlegm accumulated in lungs by adjusting a body position,
the health information acquisition unit acquires vital information which is a sensing result of a lung activity of the user as the health information, and
the processing unit determines a state of lungs of the user based on the vital information, and performs a process of obtaining the safety posture information indicating a posture suitable for the postural drainage of the user based on the posture information associated with the vital information determined to be in a third state in which the state of the lungs is good.

3. The information processing system according to claim 2, wherein the processing unit performs control of driving a movable part of at least one of a bed and a mattress used by the user within a predetermined range in a target period of acquiring the vital information and the posture information.

4. The information processing system according to claim 1, wherein, in a case where the risky posture information is obtained based on the posture information and the health information in a first period, in a second period after the first period, when the processing unit determines that the user is taking the posture corresponding to the risky posture information based on the posture information of the user, the processing unit performs notification processing.

5. The information processing system according to claim 1, wherein, in a case where the risky posture information is obtained based on the posture information and the health information in a first period, in a second period after the first period, when the processing unit determines that the user is taking the posture corresponding to the risky posture information based on the posture information of the user, the processing unit performs control of moving a movable part of at least one of a bed and a mattress used by the user so as to change the posture of the user.

6. The information processing system according to claim 1, wherein, in a case where the safety posture information is obtained based on the posture information and the health information in a first period, in a second period after the first period, when the processing unit determines that the state of the user has been worsened based on the health information of the user, the processing unit performs control of moving a movable part of at least one of a bed and a mattress used by the user so as to change the posture of the user to the posture corresponding to the safety posture information.

7. The information processing system according to claim 1, wherein
the memory stores time information indicating an acquisition timing of the posture information and the health information in association with the posture information and the health information, and
the processing unit performs control of causing a display to display a screen including a planar graph in which an axis corresponding to a value of any one of the health information, the posture information, and the time information is set as a first axis, and an axis corresponding to a value of another of the health information, the posture information, and the time information is set as a second axis intersecting the first axis, in the screen, a value of a remaining one of the health information, the posture information, and the time information being superimposed and displayed on the planar graph.

8. The information processing system according to claim 1, wherein
the memory stores time information indicating an acquisition timing of the posture information and the health information in association with the posture information and the health information, and
in a case where the risky posture information is obtained, the processing unit
determines the state of the user based on the health information acquired in a given display target period,
performs control of causing a display to display a screen including data in which the posture information and the time information are associated with each other for the health information in which the state is determined to be bad, and
performs control of causing the display to display the posture information on the screen in a mode in which an item corresponding to the risky posture information can be distinguished from items corresponding to other postures.

9. The information processing system according to claim 1, wherein
the memory stores specific risky posture information indicating a specific risky posture being a posture that can worsen the state of the user, and
the processing unit
determines whether the posture of the user indicated by the posture information is the specific risky posture based on the posture information and the specific risky posture information, and
determines a risk of the specific risky posture for the user subjected to processing by determining whether the state of the user has been worsened based on the health information associated with the posture information determined as the specific risky posture.

10. The information processing system according to claim 1, wherein
the health information acquisition unit acquires sleep information indicating a sleep state of the user as the health information, and
the processing unit
obtains a recommended sleep case based on at least one of a sleep period, a number of midway awakening, and a sleep depth specified by the sleep information, and
obtains information indicating a representative posture of the recommended sleep case as the safety posture information based on at least one of an appearance frequency and an appearance period obtained from the posture information.

11. The information processing system according to claim 1, wherein
the health information acquisition unit acquires sleep information indicating a sleep state of the user as the health information, and
the processing unit
obtains a non-recommended sleep case based on at least one of a sleep period, a number of midway awakening, and a sleep depth specified by the sleep information, and
obtains information indicating a representative posture of the non-recommended sleep case as the risky posture information based on at least one of an appearance frequency and an appearance period obtained from the posture information.

12. The information processing system according to claim 1, wherein
the health information acquisition unit acquires defecation information related to defecation of the user as the health information, and
the processing unit
obtains a representative posture information indicating a representative posture in one sleep based on at least one of an appearance frequency and an appearance period obtained from the posture information, and
specifies the representative posture information as defecation recommendation posture information indicating a posture suitable for the defecation in a case where it is determined that the defecation is present during the one sleep or on a next day based on the defecation information.

13. The information processing system according to claim 1, wherein
the health information acquisition unit acquires feedback information indicating a determination result of a physical condition by the user at get-up time as the health information, and
the processing unit
obtains a representative posture information indicating a representative posture in one sleep based on at least one of an appearance frequency and an appearance period obtained from the posture information, and
specifies the representative posture information as the risky posture information or the safety posture information in a case where it is determined that the user felt a change in the physical condition at get-up time from the one sleep based on the feedback information.

14. The information processing system according to claim 13, wherein
the feedback information is information related to nasal blockage, and
the processing unit performs at least one of
a process of determining that the representative posture information is the risky posture information having an adverse effect on the nasal blockage in a case where it is determined that the nasal blockage of the user has been worsened based on the feedback information, and
a process of determining that the representative posture information is the safety posture information having a favorable effect on the nasal blockage in a case where it is determined that the nasal blockage of the user has been improved based on the feedback information.

15. The information processing system according to claim 1, further comprising:
a movement information acquisition unit configured to acquire movement information indicating a position and a posture of a movable part of a bed or a mattress used by the user,
wherein
the memory stores the posture information, the health information, and the movement information in association with one another, and
the processing unit determines the state of the user based on the health information, and performs at least one of
(1) a process of obtaining the risky posture information based on a combination of the posture information and the movement information associated with the health information in which the state of the user is determined as a state with the worsening risk being present, and
(2) a process of obtaining the safety posture information based on a combination of the posture information and the movement information associated with the health information in which the state of the user is determined as a state with the worsening risk being relatively low.

16. The information processing system according to claim 1, wherein the posture indicated by the posture information is any one of multiple postures including at least one of a supine position, a right lateral position, a left lateral position, and a prone position.

17. The information processing system according to claim 16, wherein the processing unit obtains multiple statistic values being statistic values of the health information for each of the multiple postures, and obtains at least one of the risky posture information and the safety posture information based on comparison processing on the multiple statistic values.

18. The information processing system according to claim 1, wherein the posture information is information of the posture of the user on a bed and includes a rotation angle in a left-right direction of the user with respect to a given reference and a raise-up angle of a position of a head or feet with respect to a reference position.

19. An information processing method performed by an information processing system, the method comprising:
the information processing system acquiring posture information indicating a posture of a user,
the information processing system acquiring health information of the user,
the information processing system storing the posture information and the health information in association with each other,
the information processing system determining a state of the user based on the health information, and
the information processing system performing at least one of
(1) a process of obtaining risky posture information based on the posture information associated with the health information in which the state of the user is determined as a first state with a worsening risk being present, and
(2) a process of obtaining safety posture information based on the posture information associated with the health information in which the state of the user is determined as a second state with the worsening risk being relatively low.
